(19) United States
(12) Patent Application Publication
(10) Pub. No.: US 2002/0006410 A1
(43) Pub. Date: Jan. 17, 2002

(54) Method for treating inflammatory diseases using heat shock proteins

(75) Inventors: Katalin Veronika Lukacs, London (GB); Angela Francisca Haczku, Princeton Junction, NJ (US); Erwin W. Gelfand, Englewood, CO (US)

Correspondence Address: LEE CREWS, Fish & Richardson P.C., 225 Franklin Street, Boston, MA, 02110-2804, US

(73) Assignee: Imperial College Innovations Ltd.

(21) Appl. No.: 09/932,483

(22) Filed: Aug. 17, 2001

Related U.S. Application Data
(63) Continuation of application No. 09/012,330, filed on Jan. 23, 1998.

(51) Int. Cl. A61K 39/00; A61K 31/727; A61K 31/57

(52) U.S. Cl. 424/184.1; 514/56; 514/171; 514/251

(57) ABSTRACT

This invention relates to a method to protect a mammal from a disease associated with an inflammatory response, and in particular, from an inflammatory disease characterized by eosinophilia, airway hyperresponsiveness and/or a Th2-type immune response. The method includes administration of a heat shock protein to a mammal having such a disease. Formulations useful in the present method are also disclosed.

### FIELD OF THE INVENTION

The present invention relates to a method to protect a mammal from inflammatory diseases, and particularly, from diseases characterized by eosinophilia associated with an inflammatory response.

### BACKGROUND OF THE INVENTION

Diseases involving inflammation are characterized by the influx of certain cell types and mediators, the presence of which can lead to tissue damage and sometimes death. Diseases involving inflammation are particularly harmful when they afflict the respiratory system, resulting in obstructed breathing, hypoxemia, hypercapnia and lung tissue damage. Obstructive diseases of the airways are characterized by airflow limitation (i.e., airflow obstruction or narrowing) due to constriction of airway smooth muscle, edema and hypersecretion of mucous leading to increased work in breathing, dyspnea, hypoxemia and hypercapnia. While the mechanical properties of the lungs during obstructed breathing are shared between different types of obstructive airway disease, the pathophysiology can differ.

A variety of inflammatory agents can provoke airflow limitation including allergens, cold air, exercise, infections and air pollution. In particular, allergens and other agents in allergic or sensitized animals (i.e., antigens and haptens) cause the release of inflammatory mediators that recruit cells involved in inflammation. Such cells include lymphocytes, eosinophils, mast cells, basophils, neutrophils, macrophages, monocytes, fibroblasts and platelets. Inflammation results in airway hyperresponsiveness. A variety of studies have linked the degree, severity and timing of the inflammatory process with the degree of airway hyperresponsiveness. Thus, a common consequence of inflammation is airflow limitation and/or airway hyperresponsiveness.

Asthma is a significant disease of the lung which affects nearly 16 million Americans. Asthma is typically characterized by periodic airflow limitation and/or hyperresponsiveness to various stimuli which results in excessive airways narrowing. Other characteristics can include inflammation of airways and eosinophilia. More particularly, allergic asthma is often characterized by high IgE levels, eosinophilic airway inflammation and airway hyperresponsiveness.

Asthma prevalence (i.e., both incidence and duration) is increasing. The current prevalence approaches 10% of the population and has increased 25% in the last 20 years. Of more concern, however, is the rise in the death rate. When coupled with increases in emergency room visits and hospitalizations, recent data suggests that asthma severity is rising. While most cases of asthma are easily controlled, for those with more severe disease, the costs, the side effects and all too often, the ineffectiveness of the treatment, present serious problems. Fibroproliferative responses to chronic antigen exposure may explain both asthma severity and poor responses to therapy, especially if treatment is delayed. The majority of patients with asthma have very mild symptoms which are easily treated, but a significant number of asthmatics have more severe symptoms. Moreover, chronic asthma is associated with the development of progressive and irreversible airflow limitation due to some unknown mechanism.

Currently, therapy for treatment of inflammatory diseases such as moderate to severe asthma predominantly involves the use of immunosuppressive glucocorticosteroids. Other anti-inflammatory agents that are used to treat airway inflammation include cromolyn and nedocromil. Symptomatic treatment with beta-agonists, anticholinergic agents and methylxanthines are clinically beneficial for the relief of discomfort but fail to stop the underlying inflammatory processes that cause the disease. The frequently used systemic glucocorticosteroids have numerous side effects, including, but not limited to, weight gain, diabetes, hypertension, osteoporosis, cataracts, atherosclerosis, increased susceptibility to infection, increased lipids and cholesterol, and easy bruising. Aerosolized glucocorticosteroids have fewer side effects but can be less potent and have significant side effects, such as thrush.

Other anti-inflammatory agents, such as cromolyn and nedocromil are much less potent and have fewer side effects than glucocorticosteroids. Anti-inflammatory agents that are primarily used as immunosuppressive agents and anti-cancer agents (i.e., cytoxan, methotrexate and Immuran) have also been used to treat airway inflammation with mixed results. These agents, however, have serious side effect potential, including, but not limited to, increased susceptibility to infection, liver toxicity, drug-induced lung disease, and bone marrow suppression. Thus, such drugs have found limited clinical use for the treatment of most airway hyperresponsiveness lung diseases.

The use of anti-inflammatory and symptomatic relief reagents is a serious problem because of their side effects or their failure to attack the underlying cause of an inflammatory response. There is a continuing requirement for less harmful and more effective reagents for treating inflammation. Thus, there remains a need for processes using reagents with lower side effect profiles and less toxicity than current anti-inflammatory therapies.

### SUMMARY OF THE INVENTION

The present invention generally relates to a method to protect a mammal from a disease associated with an inflammatory response, and in particular, from a disease characterized by eosinophilia, airway hyperresponsiveness and/or a Th2-type immune response, wherein such characteristic is associated with an inflammatory response. Such a method includes the step of administering to a mammal which has such a disease, a heat shock protein. In a preferred embodiment, such a mammal is a human.

One embodiment of the present invention relates to a method to protect a mammal from a disease characterized by eosinophilia associated with an inflammatory response. The method includes the step of administering a heat shock protein to a mammal having such disease. Preferably, a such a method to treat a disease characterized by eosinophilia reduces eosinophilia in the mammal. In one embodiment, such a method reduces eosinophil blood counts in the mammal to between about 0 and about 300 cells/mm³, and more preferably, to between about 0 and about 100 cells/mm³. In another embodiment, such a method reduces eosinophil blood counts in the mammal to between about 0% and about 3% of total white blood cells in the mammal.

Diseases for which a method of the present invention can be protective include, allergic airway diseases, hyper-eosinophilic syndrome, helminthic parasitic infection, allergic rhinitis, allergic conjunctivitis, dermatitis, eczema, contact dermatitis, or food allergy. In another embodiment, the disease is a respiratory disease characterized by eosinophilic airway inflammation and airway hyperresponsiveness, such a disease including, but not limited to, allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hyper-eosinophilic syndrome, or parasitic lung disease. In another embodiment, such a disease is a disease that is associated with sensitization to an allergen, and in a preferred embodiment, is allergic asthma.

In one embodiment, a heat shock protein useful in a method of the present invention is selected from the group of an HSP-60 family heat shock protein, an HSP-70 family heat shock protein, an HSP-90 family heat shock protein, or an HSP-27 family heat shock protein. In alternate embodiments of the present method, the heat shock protein is selected from the group of an HSP-60 family heat shock protein, an HSP-70 family heat shock protein, or an HSP-27 family heat shock protein; an HSP-90 family heat shock protein or an HSP-27 family heat shock protein; or from the group of a bacterial heat shock protein and a mammalian heat shock protein. In a preferred embodiment, the heat shock protein is a mycobacterial heat shock protein, and more preferably, a mycobacterial heat shock protein-65 (HSP-65).

In some embodiments, a disease for which the present method is protective is characterized by airway hyperresponsiveness. In such embodiments, such method preferably decreases airway methacholine responsiveness in the mammal. In other embodiments, airflow limitation in the mammal is reduced such that an FEV₁ /FVC value of the mammal is at least about 80%. In another embodiment, administration of a heat shock protein results in an improvement in a mammal's PC_{20methacholine}FEV₁, value such that the PC_{20methacholine}FEV₁ value obtained before administration of a heat shock protein when the mammal is provoked with a first concentration of methacholine is the same as the PC_{20methacholine}FEV₁ value obtained after administration of the heat shock protein when the mammal is provoked with double the amount of the first concentration of methacholine. In yet another embodiment, administration of a heat shock protein improves a mammal's FEV₁ by between about 5% and about 100% of the mammal's predicted FEV₁. In another embodiment, administration of a heat shock protein reduces airflow limitation in the mammal such that an R_{L} value of the mammal is reduced by at least about 20%.

In one embodiment, a disease for which a method of the present invention is protective can be associated with increased production of a cytokine selected from the group of interleukin-4 (IL-4), interleukin-5 (IL-5), interleukin-6 (IL-6), interleukin-9 (IL-9), interleukin-10 (IL-10), interleukin-13 (IL-13) or interleukin-15 (IL-15). Accordingly, it is an embodiment of the methods of the present invention that the administration of a heat shock protein induces interferon-γ (IFN-γ) production by T lymphocytes in the mammal. In another embodiment, the administration of a heat shock protein suppresses interleukin-4 (IL-4) and interleukin-5 (IL-5) production by T lymphocytes in the mammal.

According to the methods of the present invention, a heat shock protein can be administered in an amount between about 0.1 microgram×kilogram⁻¹ and about 10 milligram×kilogram⁻¹ body weight of a mammal; and more preferably, in an amount between about 1 microgram×kilogram⁻¹ and about 1 milligram×kilogram^{31 1} body weight of a mammal. If the heat shock protein is delivered by aerosol, a heat shock protein can be administered in an amount between about 0.1 milligram×kilogram⁻¹ and about 5 milligram×kilogram⁻¹ body weight of a mammal. If the heat shock protein is delivered parenterally, a heat shock protein can be administered in an amount between about 0.1 microgram×kilogram⁻¹ and about 10 microgram×kilogram⁻¹ body weight of a mammal.

In one embodiment of the heretofore described methods of the present invention, a heat shock protein is administered in a pharmaceutically acceptable excipient. Preferred modes of administration include at least one route selected from the group of oral, nasal, topical, inhaled, transdermal, rectal or parenteral routes, and more preferably, include inhaled or nasal routes.

Another embodiment of the present invention relates to a method to protect a mammal from a disease characterized by airway hyperresponsiveness associated with an inflammatory response, the method comprising administering a heat shock protein to a mammal having such a disease. Various particular embodiments of such a method have been described above with regard to a disease characterized by eosinophilia.

Yet another embodiment of the present invention relates to a method to protect a mammal from an inflammatory disease characterized by a Th2-type immune response, the method comprising administering a heat shock protein to a mammal having such a disease. Various particular embodiments of such a method have been described above with regard to a disease characterized by eosinophilia.

Another embodiment of the present invention relates to a method for prescribing treatment for airway hyperresponsiveness or airflow limitation associated with a disease involving an inflammatory response. Such a method includes the steps of: (a) administering to a mammal's heat shock protein; (b) measuring a change in lung function in response to a provoking agent in the mammal to determine if the heat shock protein modulates airway hyperresponsiveness or airflow limitation; and, (c) prescribing a pharmacological therapy comprising administration of the heat shock protein to the mammal effective to reduce inflammation based upon the changes in lung function. In one embodiment, the step of measuring comprises measuring a value selected from the group consisting of FEV₁, FEV₁/FVC, PC_{20methacholine}FEV₁, post-enhanced h (Penh), conductance, dynamic compliance, lung resistance (R_{L}), airway pressure time index (APTI), or peak flow. A provoking agent can include a direct and an indirect stimuli, and preferably includes an agent selected from the group of an allergen, methacholine, a histamine, a leukotriene, saline, hyperventilation, exercise, sulfur dioxide, adenosine, propranolol, cold air, an antigen, bradykinin, acetylcholine, a prostaglandin, ozone, environmental air pollutants and mixtures thereof. In one embodiment of this method, the disease is characterized by airway eosinophilia.

Yet another embodiment of the present invention relates to a formulation for protecting a mammal from developing a disease characterized by eosinophilia associated with an inflammatory response, such a formulation including a heat shock protein and an anti-inflammatory agent. Such an anti-inflammatory agent can include, but is not limited to, an antigen, an allergen, a hapten, proinflammatory cytokine antagonists, proinflammatory cytokine receptor antagonists, anti-CD23, anti-IgE, leukotriene synthesis inhibitors, leukotriene receptor antagonists, glucocorticosteroids, steroid chemical derivatives, anti-cyclooxygenase agents, anti-cholinergic agents, beta-adrenergic agonists, methylxanthines, anti-histamines, cromones, zyleuton, anti-CD4 reagents, anti-IL-5 reagents, surfactants, anti-thromboxane reagents, anti-serotonin reagents, ketotiphen, cytoxin, cyclosporin, methotrexate, macrolide antibiotics, heparin, low molecular weight heparin, or mixtures thereof. In one embodiment, a formulation of the present invention includes a pharmaceutically acceptable excipient, and preferably, a pharmaceutically acceptable excipient selected from the group of biocompatible polymers, other polymeric matrices, capsules, microcapsules, microparticles, bolus preparations, osmotic pumps, diffusion devices, liposomes, lipospheres, or transdermal delivery systems.

Yet another embodiment of the present invention relates to a method to protect a mammal from a disease identified by a characteristic selected from eosinophilia, airway hyperresponsiveness and a Th2-type immune response, the characteristic being associated with an inflammatory response. This method includes the step of administering a nucleic acid molecule encoding a heat shock protein to a mammal having the disease. In a one embodiment, the nucleic acid molecule is operatively linked to a transcription control sequence. In another embodiment, the nucleic acid molecule is administered with a pharmaceutically acceptable excipient selected from the group of an aqueous physiologically balanced solution, an artificial lipid-containing substrate, a natural lipid-containing substrate, an oil, an ester, a glycol, a virus, a metal particle and a cationic molecule. In a preferred embodiment, the pharmaceutically acceptable excipient is selected from the group of liposomes, micelles, cells or cellular membranes. The nucleic acid molecule can be administered by a mode selected from the group of intradermal injection, intramuscular injection, intravenous injection, subcutaneous injection, or ex vivo administration.

### BRIEF DESCRIPTION OF THE DRAWINGS

FIG. 1A is a bar graph which demonstrates that mycobacterial HSP-65 treatment of mice during a 7 day ovalbumin-sensitization protocol upregulates non-specific and antigen-specific T cell proliferation in mice.

FIG. 2A is a line graph which shows that mycobacterial HSP-65 treatment of mice following suboptimal sensitization with ovalbumin upregulates antigen-specific T cell proliferation in the spleen.

FIG. 2B is a line graph which shows that mycobacterial HSP-65 treatment of mice following suboptimal sensitization with ovalbumin upregulates antigen-specific T cell proliferation in peribronchial lymph nodes (PBLN).

FIG. 3 is a bar graph illustrating that mycobacterial HSP-65 treatment of mice following ovalbumin sensitization and challenge upregulates both non-specific and antigen-specific T cell proliferative responses.

FIG. 4A is a bar graph showing the effect of mycobacterial HSP-65 treatment of mice following ovalbumin sensitization and challenge on production of interferon-γ by ovalbumin-stimulated splenocytes in vitro.

FIG. 4B is a bar graph showing the effect of mycobacterial HSP-65 treatment of mice following ovalbumin sensitization and challenge on production of IL-4 by ovalbumin-stimulated splenocytes in vitro.

FIG. 4C is a bar graph showing the effect of mycobacterial HSP-65 treatment of mice following ovalbumin sensitization and challenge on production of IL-5 by ovalbumin-stimulated splenocytes in vitro.

FIG. 5A is a bar graph showing the effect of mycobacterial HSP-65 treatment of mice following ovalbumin sensitization and challenge on the production of ovalbumin-specific IgG2a by ovalbumin-stimulated splenocytes in vitro.

FIG. 5B is a bar graph showing the effect of mycobacterial HSP-65 treatment of mice following ovalbumin sensitization and challenge on the production of ovalbumin-specific IgG1 by ovalbumin-stimulated splenocytes in vitro.

FIG. 5C is a bar graph showing the effect of mycobacterial HSP-65 treatment of mice following ovalbumin sensitization and challenge on the production of ovalbumin-specific IgE by ovalbumin-stimulated splenocytes in vitro.

FIG. 6 is a bar graph demonstrating that mycobacterial HSP-65 treatment of mice abolishes eosinophilic airway inflammation induced by ovalbumin sensitization and challenge in vivo.

FIG. 7 is a line graph showing that mycobacterial HSP-65 treatment of mice abolishes airway hyperresponsiveness to methacholine following ovalbumin sensitization and challenge in vivo.

### DETAILED DESCRIPTION OF THE INVENTION

The present invention generally relates to a method and formulation to protect a mammal from a disease associated with an inflammatory response, and in particular, from a disease characterized by eosinophilia, airway hyperresponsiveness and/or a Th2-type immune response, wherein such characteristic is associated with an inflammatory response. The present inventors have discovered that administration of a heat shock protein to a mammal results in significant inhibition of inflammation, and more specifically, of eosinophilia associated with inflammation. Furthermore, in respiratory diseases involving airflow limitation and/or airway hyperresponsiveness, the present inventors have discovered that administration of a heat shock protein also results in significant inhibition of airway hyperresponsiveness. Finally, the present inventors have shown that administration of heat shock protein to a mammal having an inflammatory disease characterized by a Th2-type response produces a shift (i.e., modulation) from the Th2-type immune response to a Th1-type immune response, for example, by modulating the production of cytokines and/or immunoglobulin isotypes.

Heat shock proteins are highly immunogenic proteins and have been associated with the production of various inflammatory cytokines (including both Th1- and Th2-associated cytokines, described in detail below) and with certain diseases, such as autoimmunity and of course, mycobacterial infections. Therefore, the discovery by the present inventors that administration of an immunostimulatory heat shock protein to a mammal is an effective therapeutic treatment for an inflammatory disease is surprising, particularly since current treatments for such diseases have emphasized immune suppression. Without being bound by theory, the present inventors believe that the present method of administration of a heat shock protein to protect a mammal from an inflammatory disease provides an immunostimulatory effect which results in a modulation of a harmful inflammatory immune response to an immune response that is beneficial or protective, or at least, innocuous.

According to the present invention, a heat shock protein (HSP) can be any protein belonging to a group of proteins originally identified by their increased expression in response to elevated temperatures and to other stress-related stimuli, collectively referred to in the art as “heat shock proteins”. It is now known that heat shock proteins are not only produced in response to cellular stress, but can be constitutively present in a cell and carry out various house-keeping functions.

Heat shock proteins are currently divided into at least five major families based on protein size. These five families are the HSP-100 family (i.e., having a protein size of about 100 kD); the HSP-90 family (i.e., having a protein size of about 90 kD); the HSP-70 family (i.e., having a protein size of about 70 kD); the HSP-60 family (i.e., having a protein size of about 60 kD); and the HSP-27 family (i.e., having a protein size of about 27 kD). Heat shock proteins have several unique features. For example, HSP-27, HSP-60 and HSP-70 participate in protein processing and folding and may be important in proper antigen presentation. HSP-27 and HSP-90 are known to participate in steroid binding to its receptor. Mycobacterial proteins, and particularly the mycobacterial heat shock protein-65 (HSP-65), a member of the HSP-60 heat shock family, are known to be potent inducers of cellular immune responses, and in particular, are known to enhance monocyte/macrophage and T cell functions.

A heat shock protein useful in the present invention can be a heat shock protein from any of the known heat shock families, including the above-identified heat shock protein families. Preferably, a heat shock protein useful in the present invention is from a heat shock protein family including HSP-90, HSP-70, HSP-60, and HSP-27. In one embodiment, a heat shock protein useful in the present invention is from an HSP-90 family or an HSP-27 family. In another embodiment, a heat shock protein useful in the present invention is from an HSP-60 family, an HSP-70 family, and/or an HSP-27 family. In a preferred embodiment, a heat shock protein useful in the present invention is from an HSP-60 family.

A heat shock protein useful in the present invention can be derived or obtained from any organism, preferably from a mammal or a bacteria, and even more preferably from a member of the genus Mycobacterium. Particularly preferred species of Mycobacterium from which a heat shock protein can be derived include, but are not limited to Mycobacterium tuberculosis, Mycobacterium bovis, and Mycobacterium leprae. In one embodiment, a heat shock protein useful in the present invention is a mycobacterial heat shock protein-65 (HSP-65), a 65 kD mycobacterial member of the HSP-60 family.

A heat shock protein useful in the method of the present invention can, for example, be obtained from its natural source, be produced using recombinant DNA technology, or be synthesized chemically. As used herein, a heat shock protein can be a full-length heat shock protein or any homologue of such a protein, such as a heat shock protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitation, amidation and/or addition of glycosylphosphatidyl inositol). A homologue of a heat shock protein is a protein having an amino acid sequence that is sufficiently similar to a natural heat shock protein amino acid sequence that a nucleic acid sequence encoding the homologue is capable of hybridizing under stringent conditions to (i.e., with) a nucleic acid molecule encoding the natural heat shock protein (i.e., to the complement of the nucleic acid strand encoding the natural heat shock protein amino acid sequence). A nucleic acid sequence complement of any nucleic acid sequence refers to the nucleic acid sequence of the nucleic acid strand that is complementary to (i.e., can form a complete double helix with) the strand for which the sequence is cited. Heat shock proteins useful in the method of the present invention include, but are not limited to, proteins encoded by nucleic acid molecules having full-length heat shock protein coding regions; proteins encoded by nucleic acid molecules having partial heat shock protein coding regions, wherein such proteins protect a mammal from a disease identified by a characteristic selected from eosinophilia, airway hyperresponsiveness, and/or a Th2-type immune response; fusion proteins; and chimeric proteins or chemically coupled proteins comprising combinations of different heat shock proteins, or combinations of heat shock proteins with other proteins, such as an antigen or allergen. In another embodiment, heat shock proteins useful in the method of the present invention include heat shock proteins having an amino acid sequence which is at least about 70% identical, and more preferably about 80% identical, and even more preferably, about 90% identical to the amino acid sequence of a naturally occurring heat shock protein.

The term, heat shock protein (HSP), can also refer to proteins encoded by allelic variants, including naturally occurring allelic variants of nucleic acid molecules known to encode heat shock proteins, that have similar, but not identical, nucleic acid sequences to naturally occurring, or wild-type, heat shock protein-encoding nucleic acid sequences. An allelic variant is a gene that occurs at essentially the same locus (or loci) in the genome as a heat shock protein gene, but which, due to natural variations caused by, for example, mutation or recombination, has a similar but not identical sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions).

According to the present invention, the phrase “administering a heat shock protein” can include administration of a protein directly to a mammal such as by any of the modes of administering a protein described in detail below, or alternatively, “administering a heat shock protein” can refer to administering a nucleic acid molecule encoding a heat shock protein to a mammal such that the heat shock protein is expressed in the mammal. An embodiment of the present invention in which a nucleic acid molecule encoding a heat shock protein is administered to a mammal is discussed in detail below.

According to the present invention, a heat shock protein can be administered to any member of the vertebrate class, Mammalia, including, without limitation, primates, rodents, livestock and domestic pets. Preferably, the method of the present invention is directed to the protection and/or treatment of a disease characterized by eosinophilia, airway hyperresponsiveness and/or a Th2-type response associated with an inflammatory response in mammals. A preferred mammal to protect using a heat shock protein includes a human, a rodent, a monkey, a sheep, a pig, a cat, a dog and a horse. An even more preferred mammal to protect is a human.

As used herein, the phrase “to protect a mammal from a disease” involving inflammation, refers to: reducing the potential for an inflammatory response (i.e., a response involving inflammation) to an inflammatory agent (i.e., an agent capable of causing an inflammatory response, e.g., methacholine, histamine, an allergen, a leukotriene, saline, hyperventilation, exercise, sulfur dioxide, adenosine, propranolol, cold air, an antigen or bradykinin); reducing the occurrence of the disease or inflammatory response, and/or reducing the severity of the disease or inflammatory response. Preferably, the potential for an inflammatory response is reduced, optimally, to an extent that the mammal no longer suffers discomfort and/or altered function from exposure to the inflammatory agent. For example, protecting a mammal can refer to the ability of a compound, when administered to a mammal, to prevent a disease from occurring and/or to cure or to alleviate disease symptoms, signs or causes. In particular, protecting a mammal refers to modulating an inflammatory response to suppress (e.g., reduce, inhibit or block) an overactive or harmful inflammatory response, and may include the induction of a beneficial, protective, or innocuous immune response. Also in particular, protecting a mammal refers to regulating cell-mediated immunity and/or humoral immunity (i.e., T cell activity and/or immunoglobulin activity, including Th1-type and/or Th2-type cellular and/or humoral activity). The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

A disease for which a method of the present invention is protective can include any disease characterized by eosinophilia, airway hyperresponsiveness and/or a Th2-type immune response, wherein such characteristic is associated with an inflammatory response. Such a disease can include, but is not limited to, allergic airway diseases, hyper-eosinophilic syndrome, helminthic parasitic infection, allergic rhinitis, allergic conjunctivitis, dermatitis, eczema, contact dermatitis, or food allergy. In one embodiment, a disease for which the method of the present invention can be protective includes a respiratory disease characterized by eosinophilic airway inflammation and/or airway hyperresponsiveness. Such a respiratory disease includes the above-mentioned allergic airway diseases, which can include, but are not limited to, allergic asthma, allergic bronchopulmonary aspergillosis, eosinophilic pneumonia, allergic bronchitis bronchiectasis, occupational asthma (i.e., asthma, wheezing, chest tightness and cough caused by a sensitizing agent, such as an allergen, irritant or hapten, in the work place), reactive airway disease syndrome (i.e., a single exposure to an agent that leads to asthma), and interstitial lung disease. Even more preferably, a respiratory disease for which the method of the present invention can be protective includes, but is not limited to, allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hyper-eosinophilic syndrome, and parasitic lung disease. In yet another embodiment, a disease for which the method of the present invention can be protective includes a disease that is associated with sensitization to an allergen. Examples of such diseases are described above. In a preferred embodiment, the method of the present invention protects a mammal from asthma, and particularly allergic asthma.

As discussed above, the method of the present invention protects a mammal from a disease which is characterized by eosinophilia, airway hyperresponsiveness, and/or a Th2-type immune response associated with an inflammatory response. Although each of the characteristics of eosinophilia, airway hyperresponsiveness, and a Th2-type immune response are discussed in detail separately below, it is to be understood that a method of the present invention is useful to protect a mammal from a disease having any one or a combination of these characteristics which are associated with an inflammatory response. Therefore, particular results obtained with the present method and/or further characterizations of a disease for which the method of the present invention is effective can apply to a disease having any one or a combination of the above-referenced characteristics.

One embodiment of the present invention relates to a method to protect a mammal from developing a disease characterized by eosinophilia associated with an inflammatory response. This method includes the step of administering a heat shock protein to a mammal having such a disease. As used herein, the term “eosinophilia” refers to the clinically recognized condition in which the number of eosinophils present in a mammal having eosinophilia are increased or elevated compared to the number of eosinophils present in a normal mammal (i.e., a mammal not having such a condition). In a normal mammal not having a disease characterized by eosinophilia, eosinophils typically comprise from about 0% to about 3% of the total number of white blood cells in the mammal. Eosinophil blood counts of a mammal can be measured using methods known to those of skill in the art. In particular, the eosinophil blood counts of a mammal can be measured by vital stains, such as phloxin B or Diff Quick.

According to the method of the present invention, administration of a heat shock protein to a mammal having a disease characterized by eosinophilia preferably results in a reduction in eosinophilia in the mammal. Preferably, administration of a heat shock protein in the method of the present invention reduces eosinophil blood counts in a mammal to between about 0 and 470 cells more preferably to between about 0 and 300 cells/mm³, and even more preferably to between about 0 and 100 cells/mm³. In a preferred embodiment, administration of a heat shock protein in the method of the present invention reduces eosinophil blood counts in a mammal to between about 0% and about 3% of the total number of white blood cells in a mammal.

Another embodiment of the present invention relates to a method to protect a mammal from a disease characterized by airway hyperresponsiveness associated with an inflammatory response. This method includes administering a heat shock protein to a mammal having such a disease. The term “airway hyperresponsiveness” (AHR) refers to an abnormality of the airways that allows them to narrow too easily and/or too much in response to a stimulus capable of inducing airflow limitation. AHR can be a functional alteration of the respiratory system caused by inflammation or airway remodeling (e.g., such as by collagen deposition). Airflow limitation refers to narrowing of airways that can be irreversible or reversible. Airflow limitation or airway hyperresponsiveness can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma, abnormalities in neural regulation of smooth muscle function (including adrenergic, cholinergic and nonadrenergic-noncholinergic regulation), and infiltrative diseases in and around the airways.

AHR can be measured by a stress test that comprises measuring a mammal's respiratory system function in response to a provoking agent (i.e., stimulus). AHR can be measured as a change in respiratory function from baseline plotted against the dose of a provoking agent (a procedure for such measurement and a mammal model useful therefore are described in detail below in the Examples). Respiratory function can be measured by, for example, spirometry, plethysmograph, peak flows, symptom scores, physical signs (i.e., respiratory rate), wheezing, exercise tolerance, use of rescue medication (i.e., bronchodialators) and blood gases.

In humans, spirometry can be used to gauge the change in respiratory function in conjunction with a provoking agent, such as methacholine or histamine. In humans, spirometry is performed by asking a person to take a deep breath and blow, as long, as hard and as fast as possible into a gauge that measures airflow and volume. The volume of air expired in the first second is known as forced expiratory volume (FEV₁) and the total amount of air expired is known as the forced vital capacity (FVC). In humans, normal predicted FEV₁ and FVC are available and standardized according to weight, height, sex and race. An individual free of disease has an FEV₁ and a FVC of at least about 80% of normal predicted values for a particular person and a ratio of FEV₁/FVC of at least about 80%. Values are determined before (i.e, representing a mammal's resting state) and after (i.e., representing a mammal's higher lung resistance state) inhalation of the provoking agent. The position of the resulting curve indicates the sensitivity of the airways to the provoking agent.

The effect of increasing doses or concentrations of the provoking agent on lung function can be determined by measuring the forced expired volume in 1 second (FEV₁) and FEV₁, over forced vital capacity (FEV₁/FVC ratio) of the mammal challenged with the provoking agent. In humans, the dose or concentration of a provoking agent (i.e., methacholine or histamine) that causes a 20% fall in FEV₁ (PD₂₀FEV₁) is indicative of the degree of AHR. FEV₁ and FVC values can be measured using methods known to those of skill in the art.

Pulmonary function measurements of airway resistance (R_{L}) and dynamic compliance (C_{L}) and hyperresponsiveness can be determined by measuring transpulmonary pressure as the pressure difference between the airway opening and the body plethysmograph. Volume is the calibrated pressure change in the body plethysmograph and flow is the digital differentiation of the volume signal. Resistance (R_{L}) and compliance (C_{L}) are obtained using methods known to those of skill in the art (e.g., such as by using a recursive least squares solution of the equation of motion). Airway resistance (R₁) and dynamic compliance (C₁) are described in detail in the Examples.

A variety of provoking agents are useful for measuring AHR values. Suitable provoking agent include direct and indirect stimuli. Preferred provoking agents include, for example, methacholine (Mch), histamine, an allergen, a leukotriene, saline, hyperventilation, exercise, sulfur dioxide, adenosine, propranolol, cold air, an antigen, bradykinin, acetylcholine, an environmental airborne pollutant (e.g., particulates, NO, NO₂), prostaglandins, ozone, and mixtures thereof. Preferably, methacholine is used as a provoking agent. Preferred concentrations of methacholine to use in a concentration-response curve are between about 0.001 and about 100 milligram per milliliter (mg/ml). More preferred concentrations of methacholine to use in a concentration-response curve are between about 0.01 and about 50 mg/ml. Even more preferred concentrations of methacholine to use in a concentration-response curve are between about 0.02 and about 25 mg/ml. When methacholine is used as a provoking agent, the degree of AHR is defined by the provocative concentration of methacholine needed to cause a 20% drop of the FEV₁ of a mammal (PC_{20methacholine}FEV₁). For example, in humans and using standard protocols in the art, a normal person typically has a PC_{20methacholine}FEV₁>8 mg/ml of methacholine. Thus, in humans, AHR is defined as PC_{20methacholine}FEV₁<8 mg/ml of methacholine.

The effectiveness of a drug to protect a mammal from AHR in a mammal having or susceptible to AHR is typically measured in doubling amounts. For example, the effectiveness of a drug to protect a mammal from AHR is significant if the mammal's PC_{20methacholine}FEV₁ is at 1 mg/ml before administration of the drug and is at 2 mg/ml of methacholine after administration of the drug. Similarly, a drug is considered effective if the mammal's PC_{20methacholine}FEV₁ is at 2 mg/ml before administration of the drug and is at 4 mg/ml of methacholine after administration of the drug.

In one embodiment of the present invention, a heat shock protein decreases methacholine responsiveness in a mammal. Preferably, administration of a heat shock protein increases the PC_{20methacholine}FEV₁of a mammal treated with the heat shock protein by about one doubling concentration towards the PC_{20methacholine}FEV₁of a normal mammal. A normal mammal refers to a mammal known not to suffer from or be susceptible to abnormal AHR. A test mammal refers to a mammal suspected of suffering from or being susceptible to abnormal AHR.

In another embodiment, administration of a heat shock protein to a mammal results in an improvement in a mammal's PC_{20methacholine}FEV₁ value such that the PC_{20methacholine}FEV₁ value obtained before administration of the heat shock protein when the mammal is provoked with a first concentration of methacholine is the same as the PC_{20methacholine}FEV₁ value obtained after administration of the heat shock protein when the mammal is provoked with double the amount of the first concentration of methacholine. A preferred amount of a heat shock protein to administer comprises an amount that results in an improvement in a mammal's PC_{20methacholine}FEV₁ value such that the PC_{20methacholine}FEV₁ value obtained before administration of the heat shock protein when the mammal is provoked with a concentration of methacholine that is between about 0.01 mg/ml to about 8 mg/ml, is the same as the PC_{20methacholine}FEV₁ value obtained after administration of the heat shock protein is when the mammal is provoked with a doubled concentration of methacholine of between about 0.02 mg/ml to about 16 mg/ml.

According to the present invention, respiratory function can be evaluated with a variety of static tests that comprise measuring a mammal's respiratory system function in the absence of a provoking agent. Examples of static tests include, for example, spirometry, plethysmograph, peak flows, symptom scores, physical signs (i.e., respiratory rate), wheezing, exercise tolerance, use of rescue medication (i.e., bronchodialators) and blood gases. Evaluating pulmonary function in static tests can be performed by measuring, for example, Total Lung Capacity (TLC), Thoracic Gas Volume (TgV), Functional Residual Capacity (FRC), Residual Volume (RV) and Specific Conductance (SGL) for lung volumes, Diffusing Capacity of the Lung for Carbon Monoxide (DLCO), arterial blood gases, including pH, P_{O2} and P_{CO2} for gas exchange. Both FEV₁ and FEV₁/FVC can be used to measure airflow limitation. If spirometry is used in humans, the FEV₁ of an individual can be compared to the FEV₁ of predicted values. Predicted FEV₁ values are available for standard normograms based on the mammal's age, sex, weight, height and race. A normal mammal typically has an FEV₁ at least about 80% of the predicted FEV₁ for the mammal. Airflow limitation results in a FEV₁ or FVC of less than 80% of predicted values. An alternative method to measure airflow limitation is based on the ratio of FEV₁ and FVC (FEV₁/FVC). Disease free individuals are defined as having a FEV₁/FVC ratio of at least about 80%. Airflow obstruction causes the ratio of FEV₁/FVC to fall to less than 80% of predicted values. Thus, a mammal having airflow limitation is defined by an FEV₁/FVC less than about 80%.

The effectiveness of a drug to protect a mammal having or susceptible to airflow limitation can be determined by measuring the percent improvement in FEV₁ and/or the FEV₁/FVC ratio before and after administration of the drug. In one embodiment, administration of a heat shock protein according to the present method reduces the airflow limitation of a mammal such that the FEV₁/FVC value of the mammal is at least about 80%. In another embodiment, administration of a heat shock protein improves a mammal's FEV₁ preferably by between about 5% and about 100%, more preferably by between about 6% and about 100%, more preferably by between about 7% and about 100%, and even more preferably by between about 8% and about 100% (or about 200 ml) of the mammal's predicted FEV₁.

It should be noted that measuring the airway resistance (R_{L}) value in a non-human mammal (e.g., a mouse) can be used to diagnose airflow obstruction similar to measuring the FEV₁ and/or FEV₁/FVC ratio in a human. In one embodiment of the present invention, administration of a heat shock protein reduces airflow limitation in a mammal such that an R_{L} value of the mammal is reduced by at least about 10%, and more preferably, by at least about 20%, even more preferably, by at is least about 30%, and even more preferably, by at least about 40%.

It is within the scope of the present invention that a static test can be performed before or after administration of a provocative agent used in a stress test.

In another embodiment, administration of a heat shock protein in the method of the present invention reduces the airflow limitation of a mammal such that the variation of FEV₁ or PEF values of the mammal when measured in the evening before bed and in the morning upon waking is less than about 75%, preferably less than about 45%, more preferably less than about 15%, and even more preferably less than about 8%.

Yet another embodiment of the present invention relates to a method to protect a mammal from an inflammatory disease characterized by a Th2-type immune response. This method includes administering a heat shock protein to a mammal having such a disease. According to the present invention, a disease characterized by a Th2-type immune response (alternatively referred to as a Th2 immune response), can be characterized as a disease which is associated with the predominant activation of a subset of helper T lymphocytes known in the art as Th2-type T lymphocytes (or Th2 lymphocytes), as compared to the activation of Th1-type T lymphocytes (or Th1 lymphocytes). According to the present invention, Th2-type T lymphocytes can be characterized by their production of one or more cytokines, collectively known as Th2-type cytokines. As used herein, Th2-type cytokines include interleukin-4 (IL-4), interleukin-5 (IL-5), interleukin-6 (IL-6), interleukin-9 (IL-9), interleukin-10 (IL-10), interleukin-13 (IL-13) and interleukin-15 (IL-15). In contrast, Th1-type lymphocytes produce cytokines which include IL-2 and IFN-γ. Alternatively, a Th2-type immune response can sometimes be characterized by the predominant production of antibody isotypes which include IgG1 (the approximate human equivalent of which is IgG4) and IgE, whereas a Th1-type immune response can sometimes be characterized by the production of an IgG2a or an IgG3 antibody isotype (the approximate human equivalent of which is IgG1, IgG2 or IgG3).

According to the method of the present invention, administration of a heat shock protein to a mammal having a disease characterized by a Th2-type response preferably results in a modulation of the immune response in the mammal from a Th2-type response to a more predominant Th1-type response. Preferably, administration of a heat shock protein in a method of the present invention results in a decrease (or suppression) in the production of Th2-type cytokines by T lymphocytes, such as IL-4 and IL-5. In addition, or alternatively, administration of a heat shock protein in a method of the present invention results in an increase (or induction) in the production of Th1-type cytokines by T lymphocytes, such as IFN-γ. Additionally, administration of a heat shock protein in the present method can sometimes result in a decrease in the production of Th2-type antibody isotypes, such as IgG1 and IgE, and/or an increase in the production of Th1-type antibody isotypes, such as IgG2a or IgG3.

In one embodiment, administration of a heat shock protein to a mammal having a disease as described herein preferably can reduce the level of IgG1 (the approximate equivalent human isotype of which is IgG4) in the serum of a mammal to between about 0 to about 100 international units/ml, preferably between about 0 to about 50 international units/ml, more preferably between about 0 to about 25 international units/ml, and even more preferably between about 0 to about 20 international units/ml. The concentration of IgG1 in the serum of a mammal can be measured using methods known to those of skill in the art. In particular, the concentration of IgG1 in the serum of a mammal or the concentration of IgG1 produced by B cells of a mammal in vitro can be measured by, for example, using antibodies that specifically bind to IgG1 in an enzyme-linked immunoassay or a radioimmunoassay.

In yet another embodiment, administration of a heat shock protein to a mammal having a disease as described herein preferably can increase the level of IgG2a (the approximate equivalent human isotype of which is IgG1, IgG2, or IgG3) in the serum of a mammal to between about 0 to about 100 international units/ml, preferably between about 10 to about 50 international units/ml, more preferably between about 15 to about 25 international units/ml, and even more preferably about 20 international units/ml.

As discussed above, it is an embodiment of the present invention that a Th2-type immune response can be associated with other heretofore described characteristics of a disease for which the method of the present invention is protective (e.g., eosinophilia and/or airway hyperresponsiveness). Eosinophilia, for example, is associated with production of the cytokine IL-5, and airway hyperresponsiveness can be associated with production of the cytokine, IL-4. In one embodiment of the method to protect a mammal having a disease characterized by eosinophilia, airway hyperresponsiveness and/or a Th2-type immune response associated with an inflammatory disease, such a disease can be further associated with the increased production of a cytokine selected from the group of interleukin-4 (IL-4), interleukin-5 (IL-5), interleukin-6 (IL-6), interleukin-9 (IL-9), interleukin-10 (IL-10), interleukin-13 (IL-13) and interleukin-15 (IL-15).

In accordance with the present invention, acceptable protocols for administering a heat shock protein include both the mode of administration and the amount of a heat shock protein which is to be administered to a mammal, including individual dose size, number of doses and frequency of dose administration. Determination of such protocols can be accomplished by those skilled in the art. Suitable modes of administration can include, but are not limited to, oral, nasal, topical, inhaled, transdermal, rectal, and parenteral routes. Preferred parenteral routes can include, but are not limited to, subcutaneous, intradermal, intravenous, intramuscular and intraperitoneal routes. Preferred topical routes include inhalation by aerosol (i.e., spraying), nasal administration, or topical surface administration to the skin of a mammal. In a preferred embodiment, a heat shock protein used in the method of the present invention is administered by a route selected from nasal and inhaled routes. Particularly preferred routes of administration of a nucleic acid molecule encoding a heat shock protein are discussed in detail below.

As discussed above, administration of a heat shock protein to a mammal in the method of the present invention can result in one or more effects on the mammal, which include, but are not limited to, reduction of eosinophilia (including, but not limited to, airway eosinophilic inflammation), reduction of airway hyperresponsiveness, induction of production of IFN-γ by T cells, and/or suppression of production of IL-4 and/or IL-5 by T cells. According to the method of the present invention, an effective amount of a heat shock protein to administer to a mammal comprises an amount that is capable of reducing airway hyperresponsiveness (AHR), eosinophilia, reducing airflow limitation and/or symptoms (e.g., shortness of breath, wheezing, dyspnea, exercise limitation or nocturnal awakenings), inducing production of IFN-γ by T cells, and/or suppressing production of IL-4 and/or IL-5 by T cells without being toxic to the mammal. An amount that is toxic to a mammal comprises any amount that causes damage to the structure or function of a mammal (i.e., poisonous).

A suitable single dose of a heat shock protein to administer to a mammal is a dose that is capable of protecting a mammal from a disease characterized by eosinophilia, airway hyperresponsiveness, and/or a Th2-type immune response associated with an inflammatory response when administered one or more times over a suitable time period. In particular, a suitable single dose of a heat shock protein comprises a dose that improves AHR by a doubling dose of a provoking agent or improves the static respiratory function of a mammal. Alternatively, a suitable single dose of a heat shock protein comprises a dose that reduces eosinophil counts in a mammal to the levels heretofore described, increases production of Th1-type cytokines (e.g., IFN-γ) and/or inhibits production of Th2-type cytokines (e.g., IL-4 and IL-5).

A preferred single dose of a heat shock protein comprises between about 0.1 microgram×kilogram⁻¹ and about 10 milligram×kilogram⁻¹ body weight of a mammal. A more preferred single dose of a heat shock protein comprises between about 1 microgram×kilogram⁻¹ and about 10 milligram×kilogram⁻¹ body weight of a mammal. An even more preferred single dose of a heat shock protein comprises between about 1 microgram×kilogram⁻¹ and about 5 milligram×kilogram⁻¹ body weight of a mammal. A particularly preferred single dose of a heat shock protein comprises between about 1 microgram×kilogram⁻¹ and about 1 milligram×kilograms⁻¹ body weight of a mammal. In yet another embodiment, a particularly preferred single dose of a heat shock protein comprises between about 0.1 milligram×kilogram⁻¹ and about 5 milligram×kilogram⁻¹ body weight of a mammal, if the heat shock protein is delivered by aerosol. Another particularly preferred single dose of heat shock protein comprises between about 0.1 microgram×kilogram⁻¹ and about 10 microgram×kilogram⁻¹ body weight of a mammal, if the heat shock protein is delivered parenterally.

In another embodiment, a heat shock protein of the present invention can be administered simultaneously or sequentially with a compound capable of enhancing the ability of the heat shock protein to protect a mammal from a disease characterized by eosinophilia, airway hyperresponsiveness and/or a Th2-type immune response associated with an inflammatory response. The present invention also includes a formulation containing a heat shock protein and at least one such compound to protect a mammal from a disease involving inflammation. A suitable compound to be administered simultaneously or sequentially with a heat shock protein includes a compound that is capable of regulating IgG1 or IgE production (i.e., suppression of interleukin-4 induced IgE synthesis), upregulating interferon-gamma production, regulating NK cell proliferation and activation, regulating lymphokine activated killer cells (LAK), regulating T helper cell activity, regulating degranulation of mast cells, protecting sensory nerve endings, regulating eosinophil and/or blast cell activity, preventing or relaxing smooth muscle contraction, reducing microvascular permeability or modulating Th1 and/or Th2 T cell subset differentiation. A preferred compound to be administered simultaneously or sequentially with a heat shock protein includes, including but is not limited to, any anti-inflammatory agent. According to the present invention, an anti-inflammatory agent can be any compound which is known in the art to have anti-inflammatory properties, and can also include any compound which, under certain circumstances and/or by being administered in conjunction with a heat shock protein, can provide an anti-inflammatory effect. A preferred anti-inflammatory agent to be administered simultaneously or sequentially with a heat shock protein includes, but is not limited to, an antigen, an allergen, a hapten, proinflammatory cytokine antagonists (e.g., anti-cytokine antibodies, soluble cytokine receptors), proinflammatory cytokine receptor antagonists (e.g., anti-cytokine receptor antibodies), anti-CD23, anti-IgE, leukotriene synthesis inhibitors, leukotriene receptor antagonists, glucocorticosteroids, steroid chemical derivatives, anti-cyclooxygenase agents, anti-cholinergic agents, beta-adrenergic agonists, methylxanthines, anti-histamines, cromones, zyleuton, anti-CD4 reagents, anti-IL-5 reagents, surfactants, anti-thromboxane reagents, anti-serotonin reagents, ketotiphen, cytoxin, cyclosporin, methotrexate, macrolide antibiotics, heparin, low molecular weight heparin, and mixtures thereof. The choice of compound to be administered in conjunction with a heat shock protein can be made by one of skill in the art based on various characteristics of the mammal. In particular, a mammal's genetic background, history of occurrence of inflammation, dyspnea, wheezing upon physical exam, symptom scores, physical signs (i.e., respiratory rate), exercise tolerance, use of rescue medication (i.e., bronchodialators) and blood gases.

A heat shock protein and/or formulation of the present invention to be administered to a mammal can also include other components such as a pharmaceutically acceptable excipient. For example, formulations of the present invention can be formulated in an excipient that the mammal to be protected can tolerate. Examples of such excipients include water, saline, phosphate buffered solutions, Ringer's solution, dextrose solution, Hank's solution, polyethylene glycol-containing physiologically balanced salt solutions, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability or buffers. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration. Examples of pharmaceutically acceptable excipients which are particularly useful for the administration of nucleic acid molecules encoding heat shock proteins are described in detail below.

In one embodiment of the present invention, a heat shock protein or a formulation of the present invention can include a controlled release composition that is capable of slowly releasing the heat shock protein or formulation of the present invention into a mammal. As used herein a controlled release composition comprises a heat shock protein or a formulation of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, dry powders, and transdermal delivery systems. Other controlled release compositions of the present invention include liquids that, upon administration to a mammal, form a solid or a gel in situ. Preferred controlled release compositions are biodegradable (i.e., bioerodible).

A preferred controlled release composition of the present invention is capable of releasing a heat shock protein or a formulation of the present invention into the blood of a mammal at a constant rate sufficient to attain therapeutic dose levels of a heat shock protein or the formulation to prevent inflammation over a period of time ranging from days to months based on heat shock protein toxicity parameters. A controlled release formulation of the present invention is capable of effecting protection for preferably at least about 6 hours, more preferably at least about 24 hours, and even more preferably for at least about 7 days.

Another embodiment of the present invention comprises a method for prescribing treatment for airway hyperresponsiveness and/or airflow limitation associated with a disease involving an inflammatory response, the method comprising: (1) administering to a mammal a heat shock protein; (2) measuring a change in lung function in response to a provoking agent in the mammal to determine if the heat shock protein is capable of modulating airway hyperresponsiveness and/or airflow limitation; and (3) prescribing a pharmacological therapy effective to reduce inflammation based upon the changes in lung function. In a further embodiment, such a disease is characterized by airway eosinophilia.

A change in lung function includes measuring static respiratory function before and after administration of the heat shock protein. In accordance with the present invention, the mammal receiving the heat shock protein is known to have a respiratory disease involving inflammation. Measuring a change in lung function in response to a provoking agent can be done using a variety of techniques known to those of skill in the art. Such provoking agents can include direct and indirect stimuli, and can encompass any of the heretofore mentioned provoking agents. In particular, a change in lung function can be measured by determining the FEV₁, FEV₁/FVC, PC_{20methacholine}FEV₁, post-enhanced h (Penh), conductance, dynamic compliance, lung resistance (R_{L}), airway pressure time index (APTI), and/or peak flow for the recipient of the provoking agent. Other methods to measure a change in lung function include, for example, airway resistance, dynamic compliance, lung volumes, peak flows, symptom scores, physical signs (i.e., respiratory rate), wheezing, exercise tolerance, use of rescue medication (i.e., bronchodialators) and blood gases. A suitable pharmacological therapy effective to reduce inflammation in a mammal can be evaluated by determining if and to what extent the administration of a heat shock protein has an effect on the lung function of the mammal. If a change in lung function results from the administration of a heat shock protein, then that mammal can be treated with the heat shock protein. Depending upon the extent of change in lung function, additional compounds can be administered to the mammal to enhance the treatment of the mammal. If no change or a sufficiently small change in lung function results from the administration of the heat shock protein, then that mammal should be treated with an alternative compound to the heat shock protein. The present method for prescribing treatment for a respiratory disease can also include evaluating other characteristics of the patient, such as the patient's history of respiratory disease, the presence of infectious agents, the patient's habits (e.g., smoking), the patient's working and living environment, allergies, a history of life threatening respiratory events, severity of illness, duration of illness (i.e., acute or chronic), and previous response to other drugs and/or therapy.

Another embodiment of the present invention relates to a method to protect a mammal from a disease identified by one or more characteristics selected from eosinophilia, airway hyperresponsiveness and a Th2-type immune response, wherein the characteristic is associated with an inflammatory response. This method includes the step of administering a nucleic acid molecule encoding a heat shock protein to a mammal having such a disease. Such a nucleic acid molecule encoding a heat shock protein can then be expressed by a host cell in the mammal to which the isolated nucleic acid molecule is delivered. The expressed heat shock protein can function at the site to which it is delivered in the manner as described previously herein for heat shock proteins useful in the present method (i.e., to protect a mammal from a disease characterized by eosinophilia, airway hyperresponsiveness, and/or a Th2 immune response associated with an inflammatory response).

According to the present invention, a nucleic acid molecule can include DNA, RNA, or derivatives of either DNA or RNA. A nucleic acid molecule encoding a heat shock protein can be obtained from its natural source, either as an entire (i.e., complete) gene or a portion thereof that is capable of encoding a heat shock protein that protects a mammal from a disease identified by a characteristic selected from eosinophilia, airway hyperresponsiveness, and/or a Th2-type immune response, when such protein and/or nucleic acid molecule encoding such protein is administered to the mammal. A nucleic acid molecule can also be produced using recombinant DNA technology (e.g., polymerase chain reaction (PCR) amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a heat shock protein that is useful in the method of the present invention. In one embodiment, a nucleic acid molecule encoding a heat shock protein that is useful in the present invention has a nucleic acid sequence that is at least about 70% identical, and more preferably at least about 80% identical, and even more preferably at least about 90% identical to the nucleic acid sequence of a naturally occurring heat shock protein. An isolated, or biologically pure, nucleic acid molecule, is a nucleic acid molecule that has been removed from its natural milieu. As such, “isolated” and “biologically pure” do not necessarily reflect the extent to which the nucleic acid molecule has been purified.

A nucleic acid molecule homologue can be produced using a number of methods known to those skilled in the art (see, for example, Sambrook et al., Molecular Cloning: A Laboratory Manual, Cold Spring Harbor Labs Press, 1989). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., heat shock protein activity, as appropriate). Techniques to screen for heat shock protein activity are known to those of skill in the art.

Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a heat shock protein. In addition, the phrase “recombinant molecule” primarily refers to a nucleic acid molecule operatively linked to a transcription control sequence, but can be used interchangeably with the phrase “nucleic acid molecule” which is administered to a mammal.

As described above, a nucleic acid molecule encoding a heat shock protein that is useful in a method of the present invention can be operatively linked to one or more transcription control sequences to form a recombinant molecule. The phrase “operatively linked” refers to linking a nucleic acid molecule to a transcription control sequence in a manner such that the molecule is able to be expressed when transfected (i.e., transformed, transduced or transfected) into a host cell. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in a recombinant cell useful for the expression of a heat shock protein, and/or useful to administer to a mammal in the method of the present invention. A variety of such transcription control sequences are known to those skilled in the art. Preferred transcription control sequences include those which function in mammalian, bacterial, or insect cells, and preferably in mammalian cells. More preferred transcription control sequences include, but are not limited to, simian virus 40 (SV-40), β-actin, retroviral long terminal repeat (LTR), Rous sarcoma virus (RSV), cytomegalovirus (CMV), tac, lac, trp, trc, oxy-pro, omp/lpp, rrnB, bacteriophage lambda (λ) (such as λp_{L} and λp_{R} and fusions that include such promoters), bacteriophage T7, T7 lac, bacteriophage T3, bacteriophage SP6, bacteriophage S01, metallothionein, alpha mating factor, Pichia alcohol oxidase, alphavirus subgenomic promoters (such as Sindbis virus subgenomic promoters), baculovirus, Heliothis zea insect virus, vaccinia virus and other poxviruses, herpesvirus, and adenovirus transcription control sequences, as well as other sequences capable of controlling gene expression in eukaryotic cells. Additional suitable transcription control sequences include tissue-specific promoters and enhancers (e.g., T cell-specific enhancers and promoters) . Transcription control sequences of the present invention can also include naturally occurring transcription control sequences naturally associated with a gene encoding a heat shock protein useful in a method of the present invention.

Recombinant molecules of the present invention, which can be either DNA or RNA, can also contain additional regulatory sequences, such as translation regulatory sequences, origins of replication, and other regulatory sequences that are compatible with the recombinant cell. In one embodiment, a recombinant molecule of the present invention also contains secretory signals (i.e., signal segment nucleic acid sequences) to enable an expressed heat shock protein to be secreted from a cell that produces the protein. Suitable signal segments include: (1) a bacterial signal segment, in particular a heat shock protein signal segment; or (2) any heterologous signal segment capable of directing the secretion of a heat shock protein from a cell. Preferred signal segments include, but are not limited to, signal segments naturally associated with any of the heretofore mentioned heat shock proteins.

One or more recombinant molecules of the present invention can be used to produce an encoded product (i.e., a heat shock protein). In one embodiment, an encoded product is produced by expressing a nucleic acid molecule of the present invention under conditions effective to produce the protein. A preferred method to produce an encoded protein is by transfecting a host cell with one or more recombinant molecules having a nucleic acid sequence encoding a heat shock protein to form a recombinant cell. Suitable host cells to transfect include any cell that can be transfected. Host cells can be either untransfected cells or cells that are already transformed with at least one nucleic acid molecule. Host cells of useful in the present invention can be any cell capable of producing a heat shock protein, including bacterial, fungal, mammal, and insect cells. A preferred host cell includes a mammalian cell. A more preferred host cell includes mammalian lymphocytes, muscle cells, hematopoietic precursor cells, mast cells, natural killer cells, macrophages, monocytes, epithelial cells, endothelial cells, dendritic cells, mesenchymal cells, eosinophils, lung cells, and keratinocytes.

According to the present invention, a host cell can be transfected in vivo (i.e., by delivery of the nucleic acid molecule into a mammal), ex vivo (i.e., outside of a mammal for reintroduction into the mammal, such as by introducing a nucleic acid molecule into a cell which has been removed from a mammal in tissue culture, followed by reintroduction of the cell into the mammal); or in vitro (i.e., outside of a mammal, such as in tissue culture for production of a recombinant heat shock protein). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion. Preferred methods to transfect host cells in vivo include lipofection and adsorption. A recombinant cell of the present invention comprises a host cell transfected with a nucleic acid molecule that encodes a heat shock protein. It may be appreciated by one skilled in the art that use of recombinant DNA technologies can improve expression of transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules encoding a heat shock protein include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts. The activity of an expressed recombinant heat shock protein may be improved by fragmenting, modifying, or derivatizing nucleic acid molecules encoding such a protein.

According to the present invention, a nucleic acid molecule encoding a heat shock protein can be administered, in one embodiment, with a pharmaceutically acceptable excipient. A pharmaceutically acceptable excipient can include, but is not limited to, an aqueous physiologically balanced solution, an artificial lipid-containing substrate, a natural lipid-containing substrate, an oil, an ester, a glycol, a virus, a metal particle or a cationic molecule. Particularly preferred pharmaceutically acceptable excipients for administering a nucleic acid molecule encoding a heat shock protein include liposomes, micelles, cells and cellular membranes.

Recombinant nucleic acid molecules to be administered in a method of the present invention include: (a) recombinant molecules useful in the method of the present invention in a non-targeting carrier (e.g., as “naked” DNA molecules, such as is taught, for example in Wolff et al., 1990, Science 247, 1465-1468); and (b) recombinant molecules of the present invention complexed to a delivery vehicle of the present invention. Suitable delivery vehicles for local administration comprise liposomes. Delivery vehicles for local administration can further comprise ligands for targeting the vehicle to a particular site (as described in detail herein). Preferably, a nucleic acid molecule encoding a heat shock protein is administered by a method which includes, intradermal injection, intramuscular injection, intravenous injection, subcutaneous injection, or ex vivo administration.

In one embodiment, a recombinant nucleic acid molecule useful in a method of the present invention is injected directly into muscle cells in a patient, which results in prolonged expression (e.g., weeks to months) of such a recombinant molecule. Preferably, such a recombinant molecule is in the form of “naked DNA” and is administered by direct injection into muscle cells in a patient.

A pharmaceutically acceptable excipient which is capable of targeting is herein referred to as a “delivery vehicle.” Delivery vehicles of the present invention are capable of delivering a formulation, including a heat shock protein and/or a nucleic acid molecule encoding a heat shock protein, to a target site in a mammal. A “target site” refers to a site in a mammal to which one desires to deliver a therapeutic formulation. For example, a target site can be a lung cell, an antigen presenting cell, or a lymphocyte, which is targeted by direct injection or delivery using liposomes or other delivery vehicles. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a mammal, thereby targeting and making use of a nucleic acid molecule at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a lung cell can be introduced to the outer surface of a liposome delivery vehicle so as to target the delivery vehicle to the lung cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

A preferred delivery vehicle of the present invention is a liposome. A liposome is capable of remaining stable in a mammal for a sufficient amount of time to deliver a nucleic acid molecule described in the present invention to a preferred site in the mammal. A liposome of the present invention is preferably stable in the mammal into which it has been administered for at least about 30 minutes, more preferably for at least about 1 hour and even more preferably for at least about 24 hours.

A liposome of the present invention comprises a lipid composition that is capable of targeting a nucleic acid molecule described in the present invention to a particular, or selected, site in a mammal. Preferably, the lipid composition of the liposome is capable of targeting to any organ of a mammal, more preferably to the lung, spleen, lymph nodes and skin of a mammal, and even more preferably to the lung of a mammal.

A liposome of the present invention comprises a lipid composition that is capable of fusing with the plasma membrane of the targeted cell to deliver a nucleic acid molecule into a cell. Preferably, the transfection efficiency of a liposome of the present invention is about 0.5 microgram (μg) of DNA per 16 nanomole (nmol) of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells. A preferred liposome of the present invention is between about 100 and 500 nanometers (nm), more preferably between about 150 and 450 nm and even more preferably between about 200 and 400 nm in diameter.

Suitable liposomes for use with the present invention include any liposome. Preferred liposomes of the present invention include those liposomes standardly used in, for example, gene delivery methods known to those of skill in the art. More preferred liposomes comprise liposomes having a polycationic lipid composition and/or liposomes having a cholesterol backbone conjugated to polyethylene glycol.

Complexing a liposome with a nucleic acid molecule of the present invention can be achieved using methods standard in the art (see, for example, methods described in Example 2). A suitable concentration of a nucleic acid molecule of the present invention to add to a liposome includes a concentration effective for delivering a sufficient amount of nucleic acid molecule to a cell such that the cell can produce sufficient superantigen and/or cytokine protein to regulate effector cell immunity in a desired manner. Preferably, from about 0.1 μg to about 10 μg of nucleic acid molecule of the present invention is combined with about 8 nmol liposomes, more preferably from about 0.5 μg to about 5 μg of nucleic acid molecule is combined with about 8 nmol liposomes, and even more preferably about 1.0 μg of nucleic acid molecule is combined with about 8 nmol liposomes.

Another preferred delivery vehicle comprises a recombinant virus particle vaccine. A recombinant virus particle vaccine of the present invention includes a recombinant nucleic acid molecule useful in the method of the present invention, in which the recombinant molecules are packaged in a viral coat that allows entrance of DNA into a cell so that the DNA is expressed in the cell. A number of recombinant virus particles can be used, including, but not limited to, those based on alphaviruses, poxviruses, adenoviruses, herpesviruses, arena virus and retroviruses.

The following examples are provided for the purposes of illustration and are not intended to limit the scope of the present invention.

### EXAMPLES

### Example 1

The following example demonstrates that mycobacterial heat shock protein-65 (HSP-65) upregulated T cell proliferative responses in a mouse model of airway hyperresponsiveness following short term sensitization with ovalbumin in alum.

Animal models of disease are invaluable to provide evidence to support a hypothesis or justify human experiments. Mice have many proteins which share greater than 90% homology with corresponding human proteins. For the following experiments, the present inventors have used an antigen-driven murine system that is characterized by an immune (IgE) response, a dependence on a Th2-type response, and an eosinophil response. The model is characterized by both a marked and evolving hyperresponsiveness of the airways.

The development of a versatile murine system of chronic aeroantigen exposure, which is associated with profound eosinophilia and marked, persistent and progressive airway hyperresponsiveness, provides an unparalleled opportunity to investigate potential therapeutic compositions (i.e., therapeutic formulations) for preventing or treating respiratory inflammation and/or inflammation associated with eosinophilia and a Th2-type immune response. The mouse system described herein is characterized by significant eosinophilia, followed by airway fibrosis and collagen deposition. The present inventors have used this mouse system to show that administration of the mycobacterial heat shock protein-65 (HSP-65) effectively abolishes airway hyperresponsiveness and eosinophilia in a sensitized mouse.

Female BALB/c mice between the age of 8-12 weeks were obtained from Jackson Laboratories (Bar Harbor, Me.). Mice were housed in pathogen-free conditions and were maintained on an ovalbumin (OA)-free diet. The experiments described in the following Examples were performed on age- and sex-matched groups between the age of 8-12 weeks.

To determine whether mycobacterial HSP-65 facilitates immune responses to antigenic sensitization, the effects of mycobacterial HSP-65 on T cell responses from OA-sensitized mice were studied in vitro.

In this experiment, mice were sensitized by intraperitoneal (i.p.) injection of 20 μg ovalbumin (OA) (Grade V, Sigma Chemical Co., St. Louis, Mo.) together with 20 mg alum (Al(OH)³) (Inject Alum; Pierce, Rockford, Ill.) in 100 μl PBS (phosphate-buffered saline), or with PBS alone. Immediately following the OA injection, the mice received 100 μl intravenously (i.v.) of either 100 μg of M. leprae heat shock protein-65 (mycobacterial HSP-65) in PBS (provided by Dr. Kathleen Lukacs, National Heart & Lung Institute, London) or PBS alone. 7 days later, the mice were sacrificed and the spleens were removed and placed in sterile PBS. Single-cell suspensions were prepared from the spleens, and mononuclear cells were purified by density gradient centrifugation. The cells were cultured at 2×10⁶/ml in 96-well round bottom tissue culture plates, incubating the cells in triplicate with medium alone (Med: RPMI 1640 containing heat-inactivated fetal calf serum (10%); L-glutamine (2 mM); 2-mercaptoethanol (5 mM); HEPES buffer (15 mM); penicillin (100 U/ml); and streptomycin (100 μg/ml); all components from GIBCO/BRL), with 100 μg/ml ovalbumin (OA), or with the combination of phorbol 12.13-dibutyrate (10 nM) and ionomycin (0.5 μM) (PI) for 48 hours. Cell proliferation was assessed by measuring cellular uptake of (³H)-thymidine. Cell free supernates were harvested and stored at −20° C. pending cytokine ELISA assays.

The levels of cytokine secreted into the supernates of mononuclear cell cultures were determined by ELISA. Briefly, 96-well plates (Immulon) were coated overnight (4° C.) with primary anti-cytokine capture antibody (1 μg/ml). Purified rat anti-mouse IL-4, IL-5 and IFN-γwere obtained from Pharmingen (San Diego, Calif.). The plates then were washed three times with PBS/Tween 20 (Fisher) and were blocked overnight with PBS/10% FCS. After washing, 100 μl of the cell-culture supernate samples were added to the wells. Serial dilution of standards were prepared with a dilution factor of 0.33. After overnight incubation at 4° C., the plates were washed and anti-cytokine antibodies conjugated to biotin (Pharmingen) were added at 1 μg/ml. The plates were incubated overnight and following washing 6 times, avidin-peroxidase complex (Sigma St. Louis, Mo.) and substrate were added and incubated at room temperature. A green color was developed and read at 410 nm wavelength in a spectrophotometer (Biorad 2550, Japan). The cytokine amounts were calculated by using the standard curve in each plate. The limits of detection were 5 μg/ml for IL-4 and IL-5 and 3 pg/ml for IFN-γ. As standards, recombinant mouse IL-4 (Pharmingen), IL-5 (Pharmingen) and recombinant murine IFN-γ(Genentech, San Francisco, Calif.) were used.

In order to determine antibody levels ELISA plates (Dynatech, Chantilly, Va.) were coated with OA (20 μg/ml (NaHCO₃ buffer, pH 9.6) or with polyclonal goat anti-mouse IgE 3 μg/ml (The Binding Site Ltd., San Diego, Calif.) and incubated overnight at 4° C. Plates were blocked with 0.2% gelatin buffer (pH 8.2) for 2 hours at 37° C. Standards containing OA-specific IgE and IgG were generated in the present inventor's laboratory using the method described by Oshiba et al., 1996, J. Clin. Invest. 97:1398-1408, which is incorporated herein by reference in its entirety. ELISA data were analyzed with the Microplate Manager software program for the Macintosh (Bio-Rad Labs, Richmond, Va.).

Data in all of the figures presented herein are expressed as means ± SEM. Nonparametric analysis of variance (Kruska-Wallis method) was used to determine significant variance among the groups. If a significant variance was found, the Mann-Whitney U test was used to analyze the differences between individual groups. In case of multiple comparisons, the Bonferroni correction was applied. A p value of<0.05 was considered as significant. Regression analysis was performed in order to establish correlation between variables. Data were analyzed with the MINITAB standard statistical package (Minitab Inc., State College, Pa., USA).

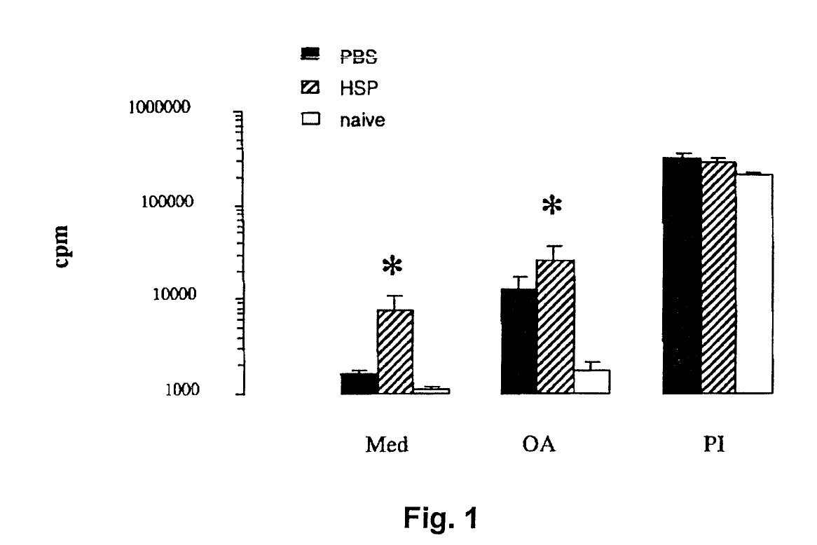

FIG. 1 shows that immunization of sensitized mice with mycobacterial HSP-65 significantly upregulated proliferative responses of splenocytes in cultures containing medium only or OA (p<0.05; n=6). Both non-specific and ovalbumin-specific proliferative responses were upregulated in mycobacterial HSP-65-treated mice. IL-4, IL-5 and IFN-γ levels as well as immunoglobulin levels were also upregulated in the culture supernates from mycobacterial HSP-65-treated mice but not in the cultures from PBS-treated mice (not shown). In summary, these data indicate that 7 days after sensitization with OA, in mice that have been immunized with mycobacterial HSP-65 but not with PBS alone, OA-dependent immune processes have been enhanced.

### Example 2

The following Example demonstrates that mycobacterial HSP-65 upregulated T cell proliferative responses in a mouse model of allergic sensitization following suboptimal sensitization with ovalbumin via aerosol challenges.

Since immunization of mice with mycobacterial HSP-65 enhanced T cell responses to OA following i.p. sensitization of mice (Example 1), the question arose as to whether mycobacterial HSP-65 would upregulate responses under conditions in which antigen-specific T cell responses would normally not be detected (i.e., suboptimal sensitization with ovalbumin). Furthermore, the following experiment was designed to test how short term mycobacterial HSP-65-treatment would affect airway responses (bronchial alveolar lavage (BAL) cellularity and airway responses to methacholine challenge).

Mice were exposed to OA aerosol (1%) on days 1, 2, 3 and 6 (suboptimal protocol), and were injected with 100 μg mycobacterial HSP-65 or PBS, i.v., on day 1 and 6. It should be noted that both immunization and subsequent antigen (OA) challenge are required to observe a response in mice in the optimal mouse model protocol. On day 7, airway responses to methacholine (MCh) were measured, bronchial alveolar lavage (BAL) samples were analyzed for their cellular content and spleens and peribronchial lymph nodes (PBLN) were removed for studying proliferative responses.

Bronchial responsiveness was assessed as a change in airway function after challenge with aerosolized methacholine via the airways using a modification of methods previously described in rats and in mice (See Haczku et al., 1995, Immunology 85:598-603; and Martin et al., 1988, J. Appl. Physiol. 64:2318-2323; both publications of which are incorporated herein by reference in their entireties). Briefly, mice were anesthetized with an intraperitoneal injection of pentobarbital sodium (70 to 90 mg/kg). A stainless steel 18 G tube was inserted as a tracheostomy cannula and was passed through a hole in the Plexiglass chamber containing the mouse. A four-way connector was attached to the tracheostomy tube, with two ports connected to the inspiratory and expiratory sides of a ventilator (model 683, Harvard Apparatus, South Natwick, Mass.). Ventilation was achieved at 160 breaths per minute and a tidal volume of 0.15 ml with a positive end-expiratory pressure of 2-4 cm H₂O. The Plexiglass chamber was continuous with a 1.0-liter glass bottle filled with copper gauze to stabilize the volume signal for thermal drift.

Transpulmonary pressure was estimated as the P_{AO}, referenced to pressure within the plethysmographic using a differential pressure transducer (Validyne Model MP-45-1-871, Validyne Engineering Corp., Northridge, Calif.). Changes in lung volume were measured by detecting pressure changes in the plethysmographic chamber referenced to pressure in a reference box using a second differential pressure transducer. The two transducers and amplifiers were electronically phased to less than 5 degrees from 1 to 30 Hz and then converted from an analog to digital signal using a 16 bit analog to digital board Model NB-MIO-16X-18 (National Instruments Corp., Austin, Tex.) at 600 bits per second per channel. The digitized signals were fed into a Macintosh Quadra 800 computer (Model M1206, Apple Computer, Inc., Cupertino, Calif.) and analyzed using the real time computer program LabVIEW (National Instruments Corp., Austin, Tex.). Flow was determined by differentiation of the volume signal and compliance was calculated as the change in volume divided by the change in pressure at zero flow points for the inspiratory phase and expiratory phase. Average compliance was calculated as the arithmetic mean of inspiratory and expiratory compliance for each breath. The LabVIEW computer program used pressure, flow, volume and average compliance to continuously calculate pulmonary resistance (R_{L}) and dynamic compliance (C_{dyn}) according to the method of Amdur et al. (pp. 364-368, 1958, Am. J. Physiol., vol. 192). The breath by breath results for R_{L}, compliance, conductance and specific compliance were tabulated and the reported values are the average of at least 10-20 breaths at the peak of response for each dose. It should be noted that measuring the R_{L} value in a mouse, can be used to diagnose airflow obstruction similar to measuring the FEV₁ and/or FEV₁/FVC ratio in a human.

The aerosolized bronchoconstrictor agents were administered through a bypass tubing via an ultrasonic nebulizer placed between the expiratory port of the ventilator and tubing via an ultrasonic nebulizer placed between the expiratory port of the ventilator and the four-way connector. Aerosolized agents were administered for 10 seconds with a tidal volume of 0.5 ml. After a dose of inhaled PBS was given, the subsequent values of R_{L} were used as a baseline. Starting 3 minutes after saline exposure, increasing concentrations of methacholine were given by inhalation (10 breaths), with the initial concentration set at 0.4 mg/ml. Increasing concentrations were given at 5-7 minute intervals. Hyperinflations of twice the tidal volume were applied between each methacholine concentration and performed by manually blocking the outflow of the ventilator in order to reverse any residual atelectasis and ensure a constant volume history prior to challenge. From twenty seconds up to three minutes after each aerosol challenge, the data of R_{L} and C_{dyn} were continuously collected and maximum values of R_{L} and C_{dyn} were taken to express changes in murine airway function.

After measurement of lung function parameters, lungs were lavaged with 1 ml aliquots of 0.9% (wt/vol) sterile NaCl (room temperature) through a polyethylene syringe attached to the tracheal cannula. Lavage fluid was centrifuged (500 ×g for 10 minutes at 4° C.), and the cell pellet was resuspended in 0.5 ml of RPMI tissue culture medium. The cell free supernatant of each BAL sample was stored at −20° C. for subsequent cytokine analysis by ELISA (described in Example 1).

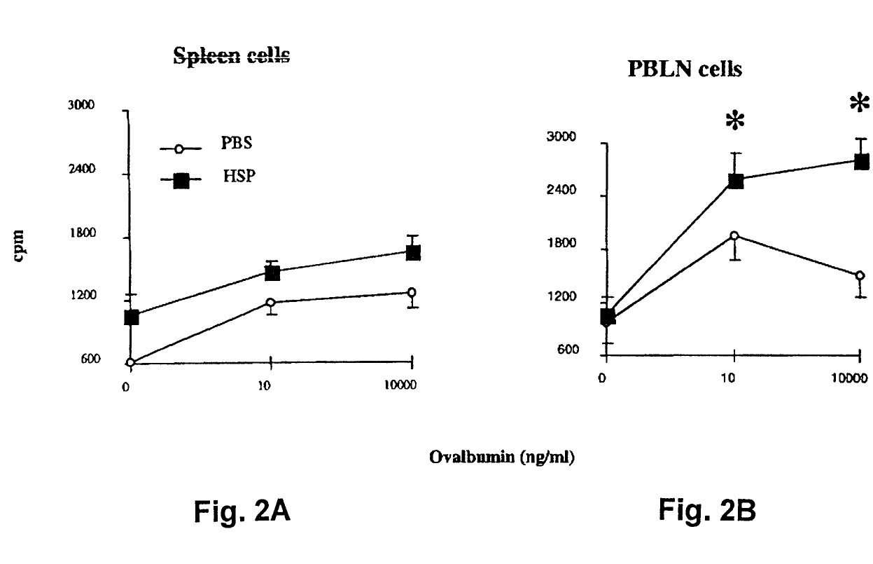

PBLN and splenocytes were analyzed by proliferation assay as described in Example 1. FIG. 2 shows that mycobacterial HSP-65 treatment, even following suboptimal sensitization with OA, significantly upregulated T cell proliferative responses to OA in both splenocytes (FIG. 2A) and peribronchial lymph node (PBLN) cells (FIG. 2B), and particularly in cells from the local draining PBLNs (p<0.05; ANOVA). No cellular changes were found in the BAL, although there was an increase in lung resistance (R_{L}) to methacholine in the group which was treated with mycobacterial HSP-65 (not shown).

These data indicate that mycobacterial HSP-65 upregulates antigen-specific immune responses even after suboptimal sensitization with OA. Further, mycobacterial HSP-65 also influences methacholine-responsiveness of the airways if given 24 hours before lung function measurements.

### Example 3

The following Example demonstrates that mycobacterial HSP-65 upregulated T cell proliferative responses in a mouse model of airway hyperresponsiveness following optimal sensitization and challenge with ovalbumin in alum.

In the mouse model of airway hyperresponsiveness and allergic sensitization used herein, it has been established that systemic sensitization and local airway challenges result in airway hyperresponsiveness (AHR) associated with eosinophilic inflammation of the airways, cardinal features of human asthma (See, for example, Bentley et al., 1992, Am. Rev. Respir. Dis. 146:500-506; Houston et al., 1953, Thorax 8:207-213; or Dunhill, 1960, J. Clin. Pathol. 13:27-33; these publications being incorporated herein by reference in their entireties). In order to investigate the effects of mycobacterial HSP-65-treatment on these pathological changes of the airways, mice were sensitized intraperitoneally with 20 μg OA (Grade V, Sigma Chemical Co., St. Louis, Mo.) together with 20 mg alum (Al(OH)³) (Inject Alum; Pierce, Rockford, Ill.) in 100 μl PBS (phosphate-buffered saline), or with PBS alone, on days 1 and 14. Mice received subsequent OA aerosol challenge for 20 min. with a 1% OA/PBS solution on days 24, 25 and 26. Mice were sacrificed and investigated 48 hr later when the peak of eosinophil infiltration and airway responses were assumed to occur.

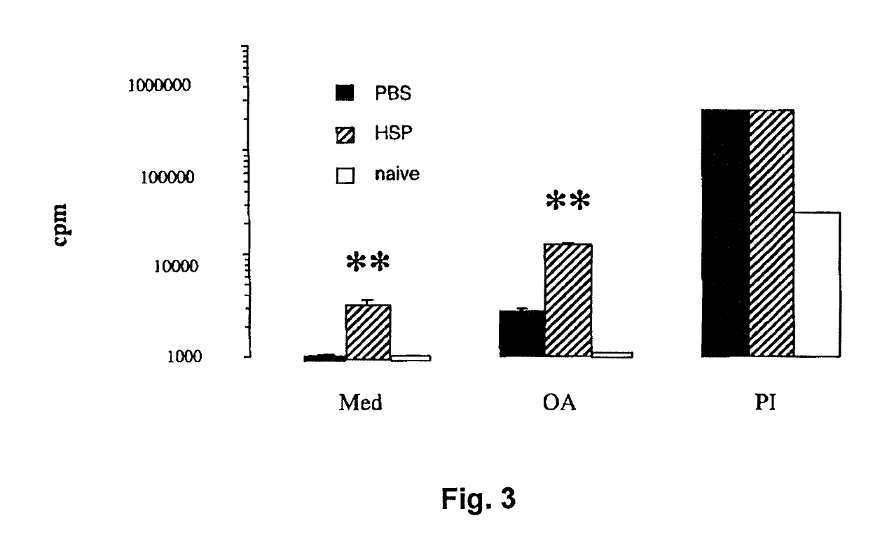

Splenic mononuclear cells from mice sensitized and challenged to OA were purified, cultured and proliferative responses to OA were assessed as described in Example 1. FIG. 3 shows that mononuclear cells from mice sensitized and challenged with OA (immunized with PBS only) showed a significant proliferative response to OA (See FIG. 3, PBS group). Further, proliferation of mononuclear cells from mycobacterial HSP-65 treated mice sensitized and challenged with OA (See FIG. 3, HSP group) was significantly enhanced in the presence of OA as well as in medium alone.

These results indicate that mononuclear cells from mycobacterial HSP-65-treated mice are activated in vivo and will display both antigen-specific and non-specific proliferation in vitro.

### Example 4

The following Example demonstrates that mycobacterial HSP-65 upregulates the production of Th1-associated cytokines and antibody isotypes, and downregulates production of Th2-associated cytokines in a mouse model of airway hyperresponsiveness following optimal sensitization and challenge with ovalbumin in alum.

Allergic asthma is characterized by high IgE levels, eosinophilic airway inflammation and airway hyperresponsiveness. T cells play a cardinal role in this disease, since upon recognition of allergen, they are capable of producing large amounts of a subset of cytokines, collectively known in the art as Th2-type cytokines. Among the Th2 cytokines, IL-4 has a unique role in inducing IgE production, and IL-5 is essential in the development of tissue eosinophilia. While production of Th1-type cytokines would normally be the consequence of T cell activation, synthesis of Th2 cytokines requires special conditions, the nature and significance of which are obscure. Without being bound by theory, the present inventors believe that allergic inflammation may reflect a pathological imbalance of Th2-versus Th1-type cytokine production, and further, such responses to common environmental antigens possibly due to the insufficiency of the regulatory mechanisms which normal operate to suppress them. The presently described murine model of airway hyperresponsiveness provided an ideal system in which to determine whether administration of heat shock protein could modulate the predominant Th2-type immune response observed in this model.

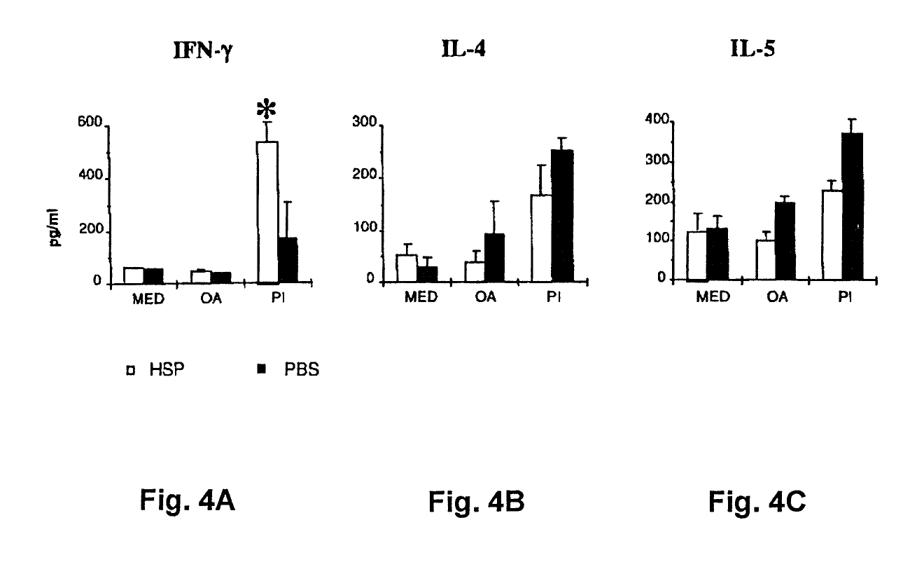

Splenic mononuclear cells from mycobacterial HSP-65- and PBS-treated mice described in Example 3 were cultured for 48 hours. The culture supernates was harvested and analyzed for cytokine release by ELISA as described in Example 1. FIG. 4 illustrates that splenocytes from mycobacterial HSP-65 treated mice produced significantly increased amount of IFN-γ (FIG. 4A) in phorbol ester/ionomycin (PI)-stimulated but not in OA-stimulated cultures, when compared with cells from PBS-treated mice (p<0.05; n=6). Meanwhile, IL-4 (FIG. 4B) and IL-5 (FIG. 4C) production in both PI and OA stimulated cultures was downregulated in splenocytes isolated from mycobacterial HSP-65-treated mice as compared to PBS-treated mice, suggesting that mycobacterial HSP-65-treatment may have a modulated effect on T cell cytokine production in vitro.

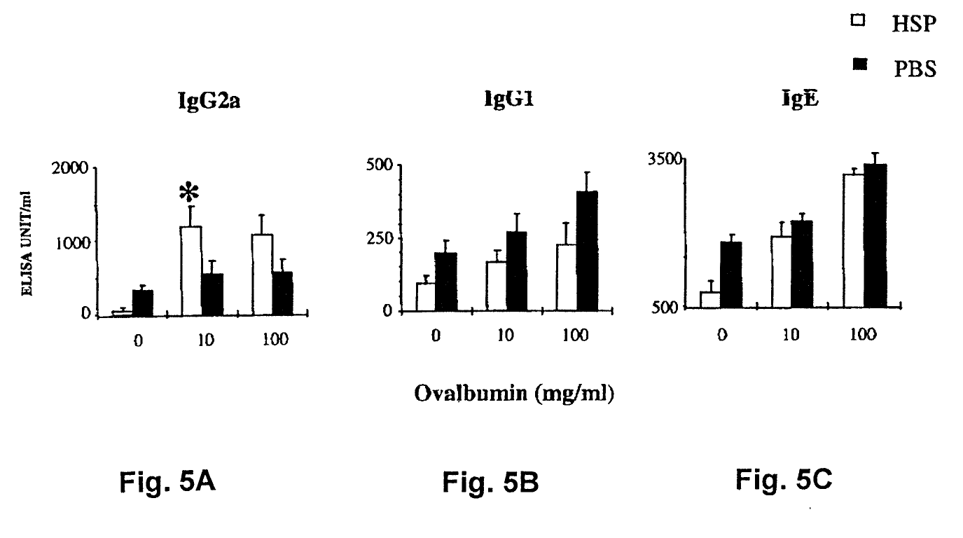

In order to assess immunoglobulin production, splenic mononuclear cells that were isolated from mice treated as described in Example 3 were cultured for 14 days in the presence of varying concentrations of OA as set forth in the X-axis of FIG. 5. Supernates were collected and analyzed for OA-specific immunoglobulin release by ELISA as described in Example 1. FIG. 5 shows that the OA-specific IgG2a production (FIG. 5A) of cells from mice treated with mycobacterial HSP-65 was significantly increased when compared with cells from PBS-treated mice (p<0.05; n=6). In vitro production of OA-specific IgG1 (FIG. 5B) and IgE (FIG. 5C) in mycobacterial HSP-65-treated mice appears to be slightly decreased compared to PBS-treated mice, although these results are not conclusive.

These data indicate that immunization of mice with mycobacterial HSP-65 modulates T cell and B cell function, and furthermore that mycobacterial HSP-65 may modulate the inflammatory immune response from a Th2 toward a Th1-type immune response.

### Example 5

The following Example demonstrates that mycobacterial HSP-65 abolises eosinophilic airway inflammation induced by sensitization and challenge with ovalbumin in a mouse model of airway hyperresponsiveness.

Allergic sensitization of the airways is associated with a massive inflammation predominated with eosinophils. In order to determine the effects of mycobacterial HSP-65 on eosinophilic airway inflammation following allergic sensitization, the cellular content of BAL was assessed in each group of mice treated as described in Example 3. Bronchial aveolar lavage was performed 48 hours after the last OA aerosol challenge as described above in Example 2. BAL cells were resuspended in RPMI and counted with a hemocytometer. Differential cell counts were made from cytospin preparations as described (See Haczku et al., supra). Cells were identified as macrophages, eosinophils, neutrophils and lymphocytes by standard morphology and at least 300 cells were counted under ×400 magnification. The percentage and absolute numbers of each cell type were then calculated.

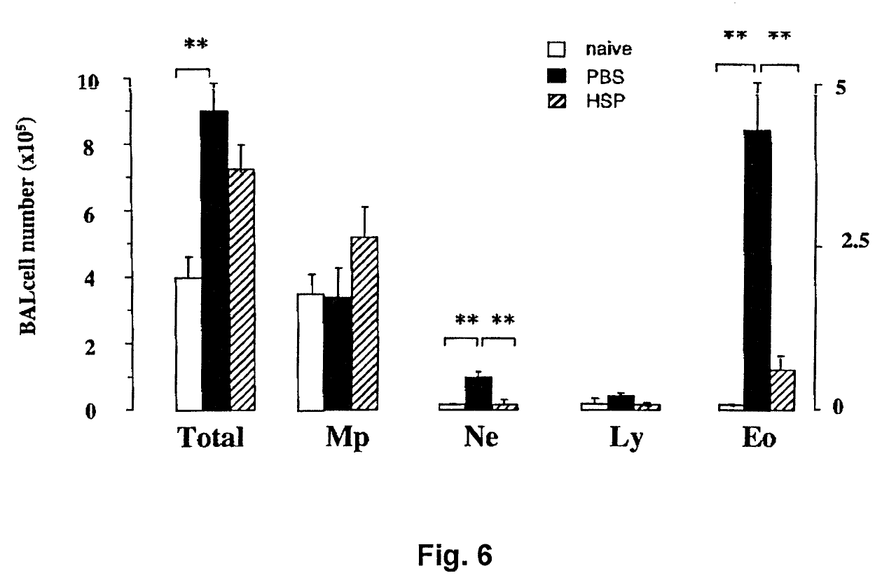

FIG. 6 shows that mice sensitized and exposed to OA and treated with PBS (normal control for airway hyperresponsiveness) developed significant airway inflammation (black bars; n=8). Approximately 60% of all the cells in the BAL consisted of eosinophils but numbers of neutrophils were also significantly increased. Naive mice (white bars; n=8) which received three days aerosol exposure to OA alone, had no eosinophils in their BAL samples. Surprisingly, no eosinophilia was detected in the mycobacterial HSP-65 treated animals (hatched bars; n=8), and these mice had a cell content that was virtually identical to the control naive mice. The difference in BAL cellular content between PBS and mycobacterial HSP-65-treated animals was significant in both the numbers of eosinophils (P<0.001) and neutrophils (P<0.001).

These results indicate that mycobacterial HSP-65 abolishes eosinophilic airway inflammation following sensitization and exposure to OA.

### Example 6

The following Example demonstrates that mycobacterial HSP-65 abolishes airway hyperresponsiveness to methacholine following sensitization and challenge of mice with ovalbumin in a mouse model of airway hyperresponsiveness.

In this experiment, bronchial responsiveness was assessed as a change in airway function after challenge with aerosolized methacholine via the airways. Mice which were treated with mycobacterial HSP-65 or PBS as described in Example 3 were anesthetized 48 hours after the final antigen challenge, cannulated and ventilated as described in Example 2. Naive mice received nebulization for three days 48 hours before their measurements were taken. Transrespiratory pressure lung volume and flow were measured, and lung resistance (R_{L}) was continuously computed, also as described in Example 2.

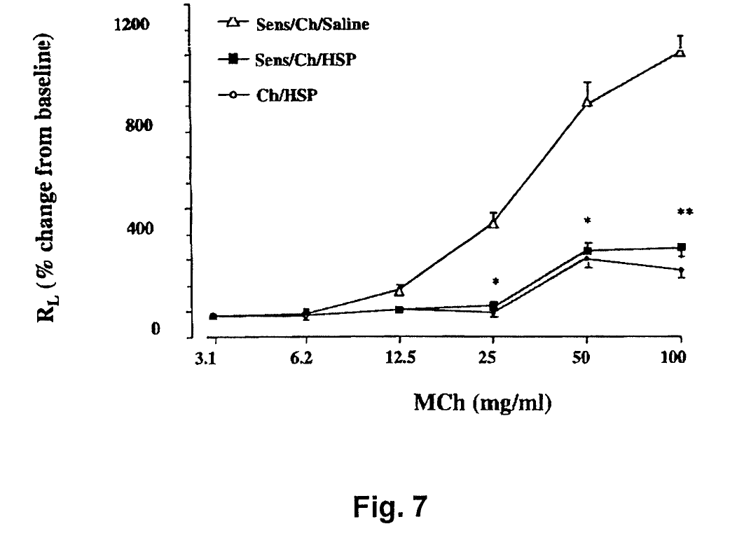

FIG. 7 illustrates that mice that were sensitized and challenged with OA and treated with PBS i.p. (normal control for airway hyperresponsiveness), demonstrated a significant increase in lung resistance (R_{L}) in response to methacholine challenge (triangles) as compared to naive mice (circles). Mice which were sensitized and challenged with OA and treated with mycobacterial HSP-65 showed normal methacholine responsiveness (squares) (i.e., almost identical to the naive mice) and significantly less than mice treated with PBS (P<0.001), indicating that mycobacterial HSP-65 treatment abolished airway hyperresponsiveness in mice sensitized with and exposed to OA.

In summary, in the above-described experiments, OA-specific immune responses were studied following in vitro culture of mononuclear cells from sensitized mice which were treated with mycobacterial HSP-65. In vivo airway responsiveness was measured by studying lung resistance to methacholine (MCh). Airway inflammation and lung tissue eosinophilia were also assessed. In mycobacterial HSP-65-treated mice, OA-specific T cell proliferation was significantly upregulated, and the supernatants of spleen cell cultures contained significantly increased IFN-γ and IgG2a. Suprisingly, the significant airway eosinophilia and heightened responsiveness to methacholine, which developed in OA sensitized and challenged mice, was abolished in mice that also received in vivo mycobacterial HSP-65 administration.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to protect a mammal from a disease characterized by eosinophilia associated with an inflammatory response, said method comprising administering a heat shock protein to a mammal having said disease.

2. The method of claim 1, wherein said disease is associated with increased production of a cytokine selected from the group consisting of interleukin-4 (IL-4), interleukin-5 (IL-5), interleukin-6 (IL-6), interleukin-9 (IL-9), interleukin-10 (IL-10), interleukin-13 (IL-13) and interleukin-15 (IL-15).

3. The method of claim 1, wherein said disease is selected from the group consisting of allergic airway diseases, hyper-eosinophilic syndrome, helminthic parasitic infection, allergic rhinitis, allergic conjunctivitis, dermatitis, eczema, contact dermatitis, and food allergy.

4. The method of claim 1, wherein said disease is a respiratory disease characterized by eosinophilic airway inflammation and airway hyperresponsiveness.

5. The method of claim 4, wherein said respiratory disease is selected from the group consisting of allergic asthma, intrinsic asthma, allergic bronchopulmonary aspergillosis, eosinophilic pneumonia, allergic bronchitis bronchiectasis, occupational asthma, reactive airway disease syndrome, interstitial lung disease, hyper-eosinophilic syndrome, and parasitic lung disease.

6. The method of claim 1, wherein said disease is associated with sensitization to an allergen.

7. The method of claim 1, wherein said disease is allergic asthma.

8. The method of claim 1, wherein said heat shock protein is selected from the group consisting of an HSP-60 family heat shock protein, an HSP-70 family heat shock protein, an HSP-90 family heat shock protein and an HSP-27 family heat shock protein.

9. The method of claim 1, wherein said heat shock protein is selected from the group consisting of an HSP-60 family heat shock protein, an HSP-70 family heat shock protein and an HSP-27 family heat shock protein.

10. The method of claim 1, wherein said heat shock protein is selected from the group consisting of an HSP-90 family heat shock protein and an HSP-27 family heat shock protein.

11. The method of claim 1, wherein said heat shock protein is selected from the group consisting of a bacterial heat shock protein and a mammalian heat shock protein.

12. The method of claim 1, wherein said heat shock protein is a mycobacterial heat shock protein.

13. The method of claim 1, wherein said heat shock protein is a mycobacterial heat shock protein-65 (HSP-65).

14. The method of claim 1, wherein said heat shock protein is administered by at least one route selected from the group consisting of oral, nasal, topical, inhaled, transdermal, rectal and parenteral routes.

15. The method of claim 1, wherein said heat shock protein is administered by a route selected from the group consisting of inhaled and nasal routes.

16. The method of claim 1, wherein said heat shock protein reduces eosinophilia in said mammal.

17. The method of claim 1, wherein said heat shock protein reduces eosinophil blood counts in said mammal to between about 0 and about 300 cells/mm³.

18. The method of claim 1, wherein said heat shock protein reduces eosinophil blood counts in said mammal to between about 0 and about 100 cells/mm³.

19. The method of claim 1, wherein said heat shock protein reduces eosinophil blood counts in said mammal to between about 0% and about 3% of total white blood cells in said mammal.

20. The method of claim 1, wherein said heat shock protein induces interferon-γ (IFN-γ) production by T lymphocytes in said mammal.

21. The method of claim 1, wherein said heat shock protein suppresses interleukin-4 (IL-4) and interleukin-5 (IL-5) production by T lymphocytes in said mammal.

22. The method of claim 1, wherein said heat shock protein decreases airway methacholine responsiveness in said mammal.

23. The method of claim 1, wherein said heat shock protein reduces airflow limitation in said mammal such that an FEV₁/FVC value of said mammal is at least about 80%.

24. The method of claim 1, wherein said heat shock protein results in an improvement in a mammal's PC_{20methacholine}FEV₁ value such that the PC_{20methacholine}FEV₁ value obtained before administration of said heat shock protein when the mammal is provoked with a first concentration of methacholine is the same as the PC_{20methacholine}FEV₁ value obtained after administration of said heat shock protein when the mammal is provoked with double the amount of the first concentration of methacholine.

25. The method of claim 24, wherein said first concentration of methacholine is between about 0.01 mg/ml and about 8 mg/ml.

26. The method of claim 1, wherein said heat shock protein improves a mammal's FEV₁ by between about 5% and about 100% of said mammal's predicted FEV₁.

27. The method of claim 1, wherein said heat shock protein reduces airflow limitation in said mammal such that an R_{L} value of said mammal is reduced by at least about 20%.

28. The method of claim 1, wherein said heat shock protein is administered in an amount between about 0.1 microgram×kilogram⁻¹ and about 10 milligram×kilograms⁻¹ body weight of a mammal.

29. The method of claim 1, wherein said heat shock protein is administered in an amount between about 1 microgram×kilogram⁻¹ and about 1 milligram×kilogram⁻¹ body weight of a mammal.

30. The method of claim 1, wherein said heat shock protein is administered in an amount between about 0.1 milligram×kilogram⁻¹ and about 5 milligram×kilogram⁻¹ body weight of a mammal, if said heat shock protein is delivered by aerosol.

31. The method of claim 1, wherein said heat shock protein is administered in an amount between about 0.1 microgram×kilogram⁻and about 10 microgram×kilogram^{31 1} body weight of a mammal, if said heat shock protein is delivered parenterally.

32. The method of claim 1, wherein said heat shock protein is administered in a pharmaceutically acceptable excipient.

33. The method of claim 1, wherein said mammal is a human.

34. A method for prescribing treatment for airway hyperresponsiveness or airflow limitation associated with a disease involving an inflammatory response, comprising:
a. administering to a mammal a heat shock protein;
b. measuring a change in lung function in response to a provoking agent in said mammal to determine if said heat shock protein modulates airway hyperresponsiveness or airflow limitation; and,
c. prescribing a pharmacological therapy comprising administration of said heat shock protein to said mammal effective to reduce inflammation based upon said changes in lung function.

35. The method of claim 34, wherein said disease is characterized by airway eosinophilia.

36. The method of claim 34, wherein said provoking agent is selected from the group consisting of a direct and an indirect stimuli.

37. The method of claim 34, wherein said provoking agent is selected from the group consisting of an allergen, methacholine, a histamine, a leukotriene, saline, hyperventilation, exercise, sulfur dioxide, adenosine, propranolol, cold air, an antigen, bradykinin, acetylcholine, a prostaglandin, ozone, environmental air pollutants and mixtures thereof.

38. The method of claim 34, wherein said step of measuring comprises measuring a value selected from the group consisting of FEV₁, FEV₁/FVC, PC_{20methacholine}FEV₁, post-enhanced h (Penh), conductance, dynamic compliance, lung resistance (R_{L}), airway pressure time index (APTI), and peak flow.

39. A method to protect a mammal from a disease characterized by airway hyperresponsiveness associated with an inflammatory response, said method comprising administering a heat shock protein to a mammal having said disease.

40. A method to protect a mammal from an inflammatory disease characterized by a Th2-type immune response, said method comprising administering a heat shock protein to a mammal having said disease.

41. A formulation for protecting a mammal from developing a disease characterized by eosinophilia associated with an inflammatory response, comprising a heat shock protein and an anti-inflammatory agent.

42. The formulation of claim 41, wherein said anti-inflammatory agent is selected from the group consisting of an antigen, an allergen, a hapten, proinflammatory cytokine antagonists, proinflammatory cytokine receptor antagonists, anti-CD23, anti-IgE, leukotriene synthesis inhibitors, leukotriene receptor antagonists, glucocorticosteroids, steroid chemical derivatives, anti-cyclooxygenase agents, anti-cholinergic agents, beta-adrenergic agonists, methylxanthines, anti-histamines, cromones, zyleuton, anti-CD4 reagents, anti-IL-5 reagents, surfactants, anti-thromboxane reagents, anti-serotonin reagents, ketotiphen, cytoxin, cyclosporin, methotrexate, macrolide antibiotics, heparin, low molecular weight heparin, and mixtures thereof.

43. The formulation of claim 41, wherein said formulation comprises a pharmaceutically acceptable excipient.

44. The formulation of claim 41, wherein said formulation comprises a pharmaceutically acceptable excipient selected from the group consisting of biocompatible polymers, other polymeric matrices, capsules, microcapsules, microparticles, bolus preparations, osmotic pumps, diffusion devices, liposomes, lipospheres, and transdermal delivery systems.

45. The method of claim 41, wherein said heat shock protein is selected from the group consisting of an HSP-60 family heat shock protein, an HSP-70 family heat shock protein, an HSP-90 family heat shock protein and an HSP-27 family heat shock protein.

46. The method of claim 41, wherein said heat shock protein is a mycobacterial heat shock protein.

47. The method of claim 41, wherein said heat shock protein is a mycobacterial heat shock protein-65 (HSP-65).

48. A method to protect a mammal from a disease identified by a characteristic selected from the group consisting of eosinophilia, airway hyperresponsiveness and a Th2-type immune response, said characteristic being associated with an inflammatory response, said method comprising administering a nucleic acid molecule encoding a heat shock protein to a mammal having said disease.

49. The method of claim 48, wherein said nucleic acid molecule is operatively linked to a transcription control sequence.

50. The method of claim 48, wherein said nucleic acid molecule is administered with a pharmaceutically acceptable excipient selected from the group consisting of an aqueous physiologically balanced solution, an artificial lipid-containing substrate, a natural lipid-containing substrate, an oil, an ester, a glycol, a virus, a metal particle and a cationic molecule.

51. The method of claim 48, wherein said pharmaceutically acceptable excipient is selected from the group consisting of liposomes, micelles, cells and cellular membranes.

52. The method of claim 48, wherein said nucleic acid molecule is administered by a mode selected from the group consisting of intradermal injection, intramuscular injection, intravenous injection, subcutaneous injection, and ex vivo administration.
